(19) United States
(12) Patent Application Publication
(10) Pub. No.: US 2026/0347870 A1
(43) Pub. Date: Nov. 5, 2026

(54) METHODS AND COMPOSITIONS FOR PPO HERBICIDE TOLERANCE

(71) Applicants: SESVanderHave NV, Tienen (BE); DLF Seeds, Roskilde (DK)

(72) Inventors: Hilde Maurice Anne VAN HOUTTE, Herent (BE); Shivegowda Shikaranahalli THAMMANNAGOWDA, Kessel-Lo (BE); Murielle Anne Francoise LOMMEL, Itterbeek (BE); Glenda WILLEMS, Beauvechain (BE); Per Erik SNELL, Landskrona (SE); Ingo Peter LENK, Roskilde (DK); Elisabeth Rosine Armand VEECKMAN, Destelbergen (BE)

(21) Appl. No.: 19/106,463

(22) PCT Filed: Sep. 1, 2023
(86) PCT No.: PCT/IB2023/058670

§ 371 Date: Feb. 25, 2025

Related U.S. Application Data
(60) Provisional application No. 63/374,316, filed on Sep. 1, 2022.
(60) Provisional application No. 63/487,737, filed on Mar. 1, 2023.

(51) Int. Cl. C12N 15/82; A01N 43/54; A01N 43/56; A01P 13/02; C07K 14/415; C12N 9/02; C12N 9/22; C12N 15/11

(52) U.S. Cl. CPC C12N 15/8274; A01N 43/54; A01N 43/56; A01P 13/02; C07K 14/415; C12N 9/001; C12N 9/226; C12N 15/11; C12N 15/8213; C12Y 103/03004; C12N 2310/20

(57) ABSTRACT

The present disclosure relates to Beta vulgaris plants having resistance to PPO herbicides and methods of producing said plants by targeted genome editing. The disclosure further relates to genetic sequences for use with targeted genome editing technologies and/or genotyping, and herbicide-resistant PPO proteins produced from genetically engineered, non-transgenic Beta vulgaris plants.

### CROSS-REFERENCE TO RELATED APPLICATIONS

This application claims the benefit of U.S. Provisional Application No. 63/374,316 filed on Sep. 1, 2022, and U.S. Provisional Application No. 63/487,737 filed on Mar. 1, 2023, both of which are hereby incorporated by reference in their entirety for all purposes.

### FIELD

The present disclosure relates to the fields of agriculture, plant biotechnology, and molecular biology. More specifically, the disclosure relates to plants and methods of producing said plants which are tolerant to herbicides that inhibit protoporphyrinogen oxidase and methods of use thereof.

### REFERENCE TO AN ELECTRONIC SEQUENCE LISTING

The contents of the electronic sequence listing (SESV_002_02WO_SeqList_ST26.xml; Size: 1,048,388 bytes; and Date of Creation: Aug. 31, 2023) are herein incorporated by reference in its entirety.

### BACKGROUND

With the increase of weed species resistant to the commonly used herbicides like glyphosate, new herbicide tolerance traits are needed in the field. Herbicides of particular interest include herbicides that inhibit protoporphyrinogen oxidase (PPO, EC 1.3.3.4), referred to as PPO herbicides. PPO herbicides provide control of a spectrum of herbicide-resistant weeds, thus making a trait conferring tolerance to these herbicides particularly useful in a cropping system. Thus, there is a need for crops having resistance to PPO herbicides. Also needed are methods for making such crops and controlling weed growth in the vicinity of such crops.

### SUMMARY OF THE DISCLOSURE

The disclosure teaches a method of producing a Beta vulgaris plant with increased tolerance to an herbicide that inhibits protoporphyrinogen oxidase comprising the steps of: a) transfecting a protoplast obtained from Beta vulgaris cells with a genome editing system to generate a transfected protoplast, wherein the genome editing system comprises: i) a Cas enzyme;
ii) at least one guide RNA (gRNA), wherein the at least one gRNA targets a genomic region corresponding to between position 5457 and 5502 of SEQ ID NO: 1; and iii) at least one single-stranded donor DNA repair template designed to introduce a deletion of glycine at a position corresponding to 208 and/or 209 of SEQ ID NO: 3; b) exposing the transfected protoplast to a selective pressure of at least one herbicide that inhibits protoporphyrinogen oxidase; c) selecting a protoplast comprising a deletion of glycine at a position corresponding to 208 and/or 209 of SEQ ID NO: 3; and d) regenerating a plant from said selected protoplast to produce a Beta vulgaris plant with increased tolerance to an herbicide that inhibits protoporphyrinogen oxidase.

The disclosure relates to a Beta vulgaris plant, or part thereof, comprising an engineered nucleic acid encoding a protoporphyrinogen oxidase 2 (PPO2) amino acid sequence, wherein said PPO2 amino acid sequence comprises a deletion of glycine at a position corresponding to 208 and/or 209 of SEQ ID NO: 3.

The disclosure further relates to a polynucleotide comprising an engineered nucleic acid sequence encoding a protein comprises a deletion of glycine at a position corresponding to 208 and/or 209.

The disclosure further teaches methods for producing a plant, plant part, or plant cell having resistance or tolerance to a PPO herbicide, the method comprising: transforming a plant, plant part, or plant cell with the polynucleotides disclosed herein.

The disclosure further teaches methods for producing a Beta vulgaris plant or plant cell having an engineered PPO2 protein comprising: a) providing a guide RNA sequence selected from SEQ ID NOs: 67-80; b) providing a donor template sequence selected from SEQ ID NOs: 48-66, and 90-96; c) providing a DNA nuclease; wherein said guide RNA, donor template, and DNA endonuclease are provided on one or more plasmids, or wherein said guide RNA and said DNA nuclease are provided as a ribonucleoprotein; d) transforming the Beta vulgaris plant or plant cell with said guide RNA, donor template, and DNA nuclease; and e) selecting a plant or plant cell having a deletion of glycine at a position corresponding to 208 and/or 209 of SEQ ID NO: 3.

The disclosure further relates to plants produced by the methods disclosed herein, and methods of using the plants for controlling undesired vegetation at a Beta vulgaris cultivation site.

The disclosure further relates to a guide RNA suitable for use in a CRISPR based genome editing system, wherein said guide RNA is selected from SEQ ID NOs: 67-80.

The disclosure further relates to a donor template sequence suitable for use in a CRISPR based genome editing system, wherein said donor template sequence is selected from SEQ ID NOs: 48-66, and 90-96.

The disclosure further relates to DNA constructs comprising the guide RNAs and donor templates disclosed herein.

The disclosure further relates to an engineered PPO2 protein comprising a deletion of glycine at a position corresponding to 208 and/or 209 in SEQ ID NO: 3.

The disclosure further teaches a method of detecting an in-frame deletion of glycine at a position corresponding to 208 and/or 209 of SEQ ID NO: 3 in a Beta vulgaris plant or part thereof, comprising: obtaining a Beta vulgaris plant or part thereof, and analyzing the Beta vulgaris plant or part thereof using at least one of SEQ ID NOs: 112-122 to detect an in-frame deletion of glycine at a position corresponding to 208 and/or 209 of SEQ ID NO: 3.

### BRIEF DESCRIPTION OF THE DRAWINGS

FIG. 1 shows the location of genetic edits in a consensus amino acid sequence for sugar beet gene PPO2 which can confer resistance to PPO herbicides.

FIG. 2 is a protein alignment produced by Clustal Omega showing the location of various edits (shaded, bold and underlined font) in SEQ ID NOs: 6, 9, 12, 15, 18, 21, 24, 27, 30, 33, 37, 40, and 43 compared to wildtype SEQ ID NO: 3.

FIG. 3 shows copies of three different edited sugar beet genotypes, HTH195, HTH240 and HTH251 heterozygous for the G208 deletion on SAF 2.5 μM containing media after 20 days.

FIG. 4 shows a chromatogram obtained after Sanger sequencing of an edited plant showing the Glycine deletion.

FIGS. 5A-5B are photographs of sugar beet plants with the expected glycine deletion at position 208/209 vs others, 21 days after spraying with Evolution, Treevix, or water. FIG. 5A shows differences in response between edited plants and non-edited (Ctrl) elite sugar beet plants 21 days after spray with Evolution 0.2×. FIG. 5B shows the phytotoxicity effect of Treevix 1×, and 2× on an elite sugar beet genotype, compared to the edited sugar beet plant HTH259 having the glycine deletion at position 208/209.

### BRIEF DESCRIPTION OF THE SEQUENCE LISTING

TABLE 1
Brief description of the sequence listing

| SEQ | Seq. | | | | | | | | | | | | Cas9 or | | |
|---|---|---|---|---|---|---|---|---|---|---|---|---|---|---|---|
| ID NO. | Type | Brief Description | Site 1 | Site 2 | OC1 | OC2 | CC1 | CC2 | AA1 | AA2 | Sil Mut OS | Sil Mut CS | Cpf1 | Lgth | +/− |
| 1 | gDNA | Genomic DNA (gDNA) | wt | wt | n.a. | n.a. | n.a. | n.a. | n.a. | n.a. | n.a. | n.a. | n.a. | 37009 | + |
| | | sequence of wild type | | | | | | | | | | | | | |
| | | Beta vulgaris PPO2 | | | | | | | | | | | | | |
| 2 | CDS | cDNA sequence of wild | wt | wt | n.a. | n.a. | n.a. | n.a. | n.a. | n.a. | n.a. | n.a. | n.a. | 1575 | + |
| | | type Beta vulgaris PPO2 | | | | | | | | | | | | | |
| 3 | protein | Protein sequence of wild | wt | wt | n.a. | n.a. | n.a. | n.a. | n.a. | n.a. | n.a. | n.a. | n.a. | 524 | + |
| | | type Beta vulgaris PPO2 | | | | | | | | | | | | | |
| 4 | gDNA | Genomic DNA (gDNA) | G208/ | n.a. | GGA | n.a. | — | n.a. | — | n.a. | n.a. | n.a. | Either | 37006 | + |
| | | sequence of dG208/209 | G209 | | | | | | | | | | | | |
| | | mutant of Beta vulgaris | | | | | | | | | | | | | |
| | | PPO2 | | | | | | | | | | | | | |
| 5 | CDS | cDNA sequence of dG208/209 | G208/ | n.a. | GGA | n.a. | — | n.a. | — | n.a. | n.a. | n.a. | Either | 1572 | + |
| | | mutant of Beta vulgaris | G209 | | | | | | | | | | | | |
| | | PPO2 | | | | | | | | | | | | | |
| 6 | protein | Protein sequence of | G208/ | n.a. | G | n.a. | n.a. | n.a. | — | n.a. | n.a. | n.a. | Either | 523 | + |
| | | dG208/209 mutant of Beta | G209 | | | | | | | | | | | | |
| | | vulgaris PPO2 | | | | | | | | | | | | | |
| 7 | gDNA | Genomic DNA (gDNA) | R126 | n.a. | AGA | n.a. | GCN | n.a. | A | n.a. | n.a. | n.a. | Cpf1 | 37009 | + |
| | | sequence of R126A | | | | | | | | | | | | | |
| | | mutant of Beta vulgaris | | | | | | | | | | | | | |
| | | PPO2 | | | | | | | | | | | | | |
| 8 | CDS | cDNA sequence of | R126 | n.a. | AGA | n.a. | GCN | n.a. | A | n.a. | n.a. | n.a. | Cpf1 | 1575 | + |
| | | R126A mutant of Beta | | | | | | | | | | | | | |
| | | vulgaris PPO2 | | | | | | | | | | | | | |
| 9 | protein | Protein sequence of | R126 | n.a. | R | n.a. | n.a. | n.a. | A | n.a. | n.a. | n.a. | Cpf1 | 524 | + |
| | | R126A mutant of Beta | | | | | | | | | | | | | |
| | | vulgaris PPO2 | | | | | | | | | | | | | |
| 10 | gDNA | Genomic DNA (gDNA) | R126 | n.a. | AGA | n.a. | GGN | n.a. | G | n.a. | n.a. | n.a. | Cpf1 | 37009 | + |
| | | sequence of R126G | | | | | | | | | | | | | |
| | | mutant of Beta vulgaris | | | | | | | | | | | | | |
| | | PPO2 | | | | | | | | | | | | | |
| 11 | CDS | cDNA sequence of | R126 | n.a. | AGA | n.a. | GGN | n.a. | G | n.a. | n.a. | n.a. | Cpf1 | 1575 | + |
| | | R126G mutant of Beta | | | | | | | | | | | | | |
| | | vulgaris PPO2 | | | | | | | | | | | | | |
| 12 | protein | Protein sequence of | R126 | n.a. | R | n.a. | n.a. | n.a. | G | n.a. | n.a. | n.a. | Cpf1 | 524 | + |
| | | R126G mutant of Beta | | | | | | | | | | | | | |
| | | vulgaris PPO2 | | | | | | | | | | | | | |
| 13 | gDNA | Genomic DNA (gDNA) | R126 | n.a. | AGA | n.a. | CTN | n.a. | L | n.a. | n.a. | n.a. | Either | 37009 | + |
| | | sequence of R126L | | | | | | | | | | | | | |
| | | mutant of Beta vulgaris | | | | | | | | | | | | | |
| | | PPO2 | | | | | | | | | | | | | |
| 14 | CDS | cDNA sequence of | R126 | n.a. | AGA | n.a. | CTN | n.a. | L | n.a. | n.a. | n.a. | Either | 1575 | + |
| | | R126L mutant of Beta | | | | | | | | | | | | | |
| | | vulgaris PPO2 | | | | | | | | | | | | | |
| 15 | protein | Protein sequence of | R126 | n.a. | R | n.a. | n.a. | n.a. | L | n.a. | n.a. | n.a. | Either | 524 | + |
| | | R126L mutant of Beta | | | | | | | | | | | | | |
| | | vulgaris PPO2 | | | | | | | | | | | | | |
| 16 | gDNA | Genomic DNA (gDNA) | R126 | n.a. | AGA | n.a. | ATH | n.a. | I | n.a. | n.a. | n.a. | Cpf1 | 37009 | + |
| | | sequence of R126I mutant | | | | | | | | | | | | | |
| | | of Beta vulgaris PPO2 | | | | | | | | | | | | | |
| 17 | CDS | cDNA sequence of R126I | R126 | n.a. | AGA | n.a. | ATH | n.a. | I | n.a. | n.a. | n.a. | Cpf1 | 1575 | + |
| | | mutant of Beta vulgaris | | | | | | | | | | | | | |
| | | PPO2 | | | | | | | | | | | | | |
| 18 | protein | Protein sequence of | R126 | n.a. | R | n.a. | n.a. | n.a. | I | n.a. | n.a. | n.a. | Cpf1 | 524 | + |
| | | R126I mutant of Beta | | | | | | | | | | | | | |
| | | vulgaris PPO2 | | | | | | | | | | | | | |
| 19 | gDNA | Genomic DNA (gDNA) | R126 | n.a. | AGA | n.a. | ATG | n.a. | M | n.a. | n.a. | n.a. | Cpf1 | 37009 | + |
| | | sequence of R126M | | | | | | | | | | | | | |
| | | mutant of Beta vulgaris | | | | | | | | | | | | | |
| | | PPO2 | | | | | | | | | | | | | |
| 20 | CDS | cDNA sequence of | R126 | n.a. | AGA | n.a. | ATG | n.a. | M | n.a. | n.a. | n.a. | Cpf1 | 1575 | + |
| | | R126M mutant of Beta | | | | | | | | | | | | | |
| | | vulgaris PPO2 | | | | | | | | | | | | | |
| 21 | protein | Protein sequence of | R126 | n.a. | R | n.a. | n.a. | n.a. | M | n.a. | n.a. | n.a. | Cpf1 | 524 | + |
| | | R126M mutant of Beta | | | | | | | | | | | | | |
| | | vulgaris PPO2 | | | | | | | | | | | | | |
| 22 | gDNA | Genomic DNA (gDNA) | F420 | n.a. | TTT | n.a. | GTN | n.a. | V | n.a. | n.a. | n.a. | Cpf1 | 37009 | + |
| | | sequence of F420V | | | | | | | | | | | | | |
| | | mutant of Beta vulgaris | | | | | | | | | | | | | |
| | | PPO2 | | | | | | | | | | | | | |
| 23 | CDS | cDNA sequence of | F420 | n.a. | TTT | n.a. | GTN | n.a. | V | n.a. | n.a. | n.a. | Cpf1 | 1575 | + |
| | | F420V mutant of Beta | | | | | | | | | | | | | |
| | | vulgaris PPO2 | | | | | | | | | | | | | |
| 24 | protein | Protein sequence of | F420 | n.a. | F | n.a. | n.a. | n.a. | V | n.a. | n.a. | n.a. | Cpf1 | 524 | + |
| | | F420V mutant of Beta | | | | | | | | | | | | | |
| | | vulgaris PPO2 | | | | | | | | | | | | | |
| 25 | gDNA | Genomic DNA (gDNA) | F420 | n.a. | TTT | n.a. | ATG | n.a. | M | n.a. | n.a. | n.a. | Cpf1 | 37009 | + |
| | | sequence of F420M | | | | | | | | | | | | | |
| | | mutant of Beta vulgaris | | | | | | | | | | | | | |
| | | PPO2 | | | | | | | | | | | | | |
| 26 | CDS | cDNA sequence of | F420 | n.a. | TTT | n.a. | ATG | n.a. | M | n.a. | n.a. | n.a. | Cpf1 | 1575 | + |
| | | F420M mutant of Beta | | | | | | | | | | | | | |
| | | vulgaris PPO2 | | | | | | | | | | | | | |
| 27 | protein | Protein sequence of | F420 | n.a. | F | n.a. | n.a. | n.a. | M | n.a. | n.a. | n.a. | Cpf1 | 524 | + |
| | | F420M mutant of Beta | | | | | | | | | | | | | |
| | | vulgaris PPO2 | | | | | | | | | | | | | |
| 28 | gDNA | Genomic DNA (gDNA) | F420 | n.a. | TTT | n.a. | ATH | n.a. | I | n.a. | n.a. | n.a. | Cpf1 | 37009 | + |
| | | sequence of F420I mutant | | | | | | | | | | | | | |
| | | of Beta vulgaris PPO2 | | | | | | | | | | | | | |
| 29 | CDS | cDNA sequence of F420I | F420 | n.a. | TTT | n.a. | ATH | n.a. | I | n.a. | n.a. | n.a. | Cpf1 | 1575 | + |
| | | mutant of Beta vulgaris | | | | | | | | | | | | | |
| | | PPO2 | | | | | | | | | | | | | |
| 30 | protein | Protein sequence of F420I | F420 | n.a. | F | n.a. | n.a. | n.a. | I | n.a. | n.a. | n.a. | Cpf1 | 524 | + |
| | | mutant of Beta vulgaris | | | | | | | | | | | | | |
| | | PPO2 | | | | | | | | | | | | | |
| 31 | gDNA | Genomic DNA (gDNA) | F420 | n.a. | TTT | n.a. | CTN | n.a. | L | n.a. | n.a. | n.a. | Cpf1 | 37009 | + |
| | | sequence of F420L | | | | | | | | | | | | | |
| | | mutant of Beta vulgaris | | | | | | | | | | | | | |
| | | PPO2 | | | | | | | | | | | | | |
| 32 | CDS | cDNA sequence of F420L | F420 | n.a. | TTT | n.a. | CTN | n.a. | L | n.a. | n.a. | n.a. | Cpf1 | 1575 | + |
| | | mutant of Beta vulgaris | | | | | | | | | | | | | |
| | | PPO2 | | | | | | | | | | | | | |
| 33 | protein | Protein sequence of | F420 | n.a. | F | n.a. | n.a. | n.a. | L | n.a. | n.a. | n.a. | Cpf1 | 524 | + |
| | | F420L mutant of Beta | | | | | | | | | | | | | |
| | | vulgaris PPO2 | | | | | | | | | | | | | |
| 34 | CDS | CDS for codon optimized | n.a. | n.a. | n.a. | n.a. | n.a. | n.a. | n.a. | n.a. | n.a. | n.a. | Cpf1 | 4272 | + |
| | | Cpf1 | | | | | | | | | | | | | |
| 35 | gDNA | Genomic DNA (gDNA) | L397 | n.a. | CTC | n.a. | GAR | n.a. | E | n.a. | n.a. | n.a. | Cas9 | 37009 | + |
| | | sequence of L397E | | | | | | | | | | | | | |
| | | mutant of Beta vulgaris | | | | | | | | | | | | | |
| | | PPO2 | | | | | | | | | | | | | |
| 36 | CDS | cDNA sequence of L397E | L397 | n.a. | CTC | n.a. | GAR | n.a. | E | n.a. | n.a. | n.a. | Cas9 | 1575 | + |
| | | mutant of Beta vulgaris | | | | | | | | | | | | | |
| | | PPO2 | | | | | | | | | | | | | |
| 37 | protein | Protein sequence of | L397 | n.a. | L | n.a. | n.a. | n.a. | E | n.a. | n.a. | n.a. | Cas9 | 524 | + |
| | | L397E mutant of Beta | | | | | | | | | | | | | |
| | | vulgaris PPO2 | | | | | | | | | | | | | |
| 38 | gDNA | Genomic DNA (gDNA) | G398 | n.a. | GGT | n.a. | GCT | n.a. | A | n.a. | n.a. | n.a. | Cas9 | 37009 | + |
| | | sequence of G398A | | | | | | | | | | | | | |
| | | mutant of Beta vulgaris | | | | | | | | | | | | | |
| | | PPO2 | | | | | | | | | | | | | |
| 39 | CDS | cDNA sequence of | G398 | n.a. | GGT | n.a. | GCT | n.a. | A | n.a. | n.a. | n.a. | Cas9 | 1575 | + |
| | | G398A mutant of Beta | | | | | | | | | | | | | |
| | | vulgaris PPO2 | | | | | | | | | | | | | |
| 40 | protein | Protein sequence of | G398 | n.a. | G | n.a. | n.a. | n.a. | A | n.a. | n.a. | n.a. | Cas9 | 524 | + |
| | | G398A mutant of Beta | | | | | | | | | | | | | |
| | | vulgaris PPO2 | | | | | | | | | | | | | |
| 41 | gDNA | Genomic DNA (gDNA) | L397 | F420 | CTC | TTT | GAR | GTN | E | V | n.a. | n.a. | Cas9 | 37009 | + |
| | | sequence of | | | | | | | | | | | | | |
| | | L397E + F420V double | | | | | | | | | | | | | |
| | | mutant of Beta vulgaris | | | | | | | | | | | | | |
| | | PPO2 | | | | | | | | | | | | | |
| 42 | CDS | cDNA sequence of | L397 | F420 | CTC | TTT | GAR | GTN | E | V | n.a. | n.a. | Cas9 | 1575 | + |
| | | L397E + F420V double | | | | | | | | | | | | | |
| | | mutant of Beta vulgaris | | | | | | | | | | | | | |
| | | PPO2 | | | | | | | | | | | | | |
| 43 | protein | Protein sequence of | L397 | F420 | L | F | n.a. | n.a. | E | V | n.a. | n.a. | Cas9 | 524 | + |
| | | L397E + F420V double | | | | | | | | | | | | | |
| | | mutant of Beta vulgaris | | | | | | | | | | | | | |
| | | PPO2 | | | | | | | | | | | | | |
| 44 | gDNA | Genomic DNA (gDNA) | R126 | n.a. | AGA | n.a. | TTR | n.a. | L | n.a. | n.a. | n.a. | Either | 37009 | + |
| | | sequence of R126L | | | | | | | | | | | | | |
| | | mutant of Beta vulgaris | | | | | | | | | | | | | |
| | | PPO2 | | | | | | | | | | | | | |
| 45 | CDS | cDNA sequence of | R126 | n.a. | AGA | n.a. | TTR | n.a. | L | n.a. | n.a. | n.a. | Either | 1575 | + |
| | | R126L mutant of Beta | | | | | | | | | | | | | |
| | | vulgaris PPO2 | | | | | | | | | | | | | |
| 46 | gDNA | Genomic DNA (gDNA) | F420 | n.a. | TTT | n.a. | TTR | n.a. | L | n.a. | n.a. | n.a. | Cpf1 | 37009 | + |
| | | sequence of F420L | | | | | | | | | | | | | |
| | | mutant of Beta vulgaris | | | | | | | | | | | | | |
| | | PPO2 | | | | | | | | | | | | | |
| 47 | CDS | cDNA sequence of F420L | F420 | n.a. | TTT | n.a. | TTR | n.a. | L | n.a. | n.a. | n.a. | Cpf1 | 1575 | + |
| | | mutant of Beta vulgaris | | | | | | | | | | | | | |
| | | PPO2 | | | | | | | | | | | | | |
| 48 | donor | Donor template for | F420 | n.a. | TTT | n.a. | ATG | n.a. | M | n.a. | n.a. | n.a. | Cpf1 | 251 | − |
| | | F420M | | | | | | | | | | | | | |
| 49 | donor | Donor template for | F420 | n.a. | TTT | n.a. | ATH | n.a. | I | n.a. | n.a. | n.a. | Cpf1 | 251 | − |
| | | F420I | | | | | | | | | | | | | |
| 50 | donor | Donor template for | F420 | n.a. | TTT | n.a. | CTN | n.a. | L | n.a. | n.a. | n.a. | Cpf1 | 251 | − |
| | | F420L | | | | | | | | | | | | | |
| 51 | donor | Donor template for | G208/ | n.a. | GGA | n.a. | — | n.a. | — | n.a. | n.a. | n.a. | Cpf1 | 262 | − |
| | | dG208/209 | G209 | | | | | | | | | | | | |
| 52 | donor | Donor template for | R126 | n.a. | AGA | n.a. | GCN | n.a. | A | n.a. | n.a. | n.a. | Cpf1 | 335 | − |
| | | R126A | | | | | | | | | | | | | |
| 53 | donor | Donor template for | R126 | n.a. | AGA | n.a. | GGN | n.a. | G | n.a. | n.a. | n.a. | Cpf1 | 335 | − |
| | | R126G | | | | | | | | | | | | | |
| 54 | donor | Donor template for | R126 | n.a. | AGA | n.a. | CTN | n.a. | L | n.a. | n.a. | n.a. | Cpf1 | 335 | − |
| | | R126L | | | | | | | | | | | | | |
| 55 | donor | Donor template for | R126 | n.a. | AGA | n.a. | ATH | n.a. | I | n.a. | n.a. | n.a. | Cpf1 | 335 | − |
| | | R126I | | | | | | | | | | | | | |
| 56 | donor | Donor template for | R126 | n.a. | AGA | n.a. | ATG | n.a. | M | n.a. | n.a. | n.a. | Cpf1 | 335 | − |
| | | R126M | | | | | | | | | | | | | |
| 57 | donor | Donor template for | F420 | n.a. | TTT | n.a. | GTN | n.a. | V | n.a. | n.a. | n.a. | Cpf1 | 251 | − |
| | | F420V | | | | | | | | | | | | | |
| 58 | donor | Donor template for | R126 | n.a. | AGA | n.a. | TTR | n.a. | L | n.a. | n.a. | n.a. | Either | 161 | + |
| | | R126L (for Cas9-Cpf1) | | | | | | | | | | | | | |
| 59 | donor | Donor template for | R126 | n.a. | AGA | n.a. | CTN | n.a. | L | n.a. | n.a. | n.a. | Either | 161 | + |
| | | R126L (for Cas9-Cpf1) | | | | | | | | | | | | | |
| 60 | donor | Donor template for G208/209 | G208/ | n.a. | GGA | n.a. | — | n.a. | — | n.a. | n.a. | n.a. | Cas9 | 175 | + |
| | | (for Cas9) | G209 | | | | | | | | | | | | |
| 61 | donor | Donor template for | G398 | n.a. | GGT | n.a. | GCN | n.a. | A | n.a. | n.a. | n.a. | Either | 155 | − |
| | | G398A (for Cas9-Cpf1) | | | | | | | | | | | | | |
| 62 | donor | Donor template L397E | L397 | F420 | CTC | TTT | GAR | GTN | E | V | n.a. | n.a. | Cas9 | 200 | + |
| | | and F420V (for Cas9) | | | | | | | | | | | | | |
| 63 | donor | Donor template for G208/209 | G208/ | n.a. | GGA | n.a. | — | n.a. | — | n.a. | n.a. | n.a. | Cpf1 | 154 | − |
| | | (for Cpf1) | G209 | | | | | | | | | | | | |
| 64 | donor | Donor template for | L397 | F420 | CTC | TTT | GAR | GTN | E | V | n.a. | n.a. | Cpf1 | 200 | − |
| | | L397E and F420V (for | | | | | | | | | | | | | |
| | | Cpf1) | | | | | | | | | | | | | |
| 65 | donor | Donor template for | F420 | n.a. | TTT | n.a. | TTR | n.a. | L | n.a. | n.a. | n.a. | Cpf1 | 251 | − |
| | | F420L | | | | | | | | | | | | | |
| 66 | donor | Donor template for | R126 | n.a. | AGA | n.a. | TTR | n.a. | L | n.a. | n.a. | n.a. | Cpf1 | 335 | − |
| | | R126L | | | | | | | | | | | | | |
| 67 | guide | sgRNA1 for L397E and | L397 | F420 | n.a. | n.a. | n.a. | n.a. | n.a. | n.a. | n.a. | n.a. | Cas9 | 20 | + |
| | | F420V (for Cas9) | | | | | | | | | | | | | |
| 68 | guide | sgRNA2 for L397E and | L397 | F420 | n.a. | n.a. | n.a. | n.a. | n.a. | n.a. | n.a. | n.a. | Cas9 | 20 | + |
| | | F420V (for Cas9) | | | | | | | | | | | | | |
| 69 | guide | sgRNA for R126L (for | R126 | n.a. | n.a. | n.a. | n.a. | n.a. | n.a. | n.a. | n.a. | n.a. | Cpf1 | 20 | − |
| | | Cpf1) | | | | | | | | | | | | | |
| 70 | guide | sgRNA1 for R126 | R126 | n.a. | n.a. | n.a. | n.a. | n.a. | n.a. | n.a. | n.a. | n.a. | Cpf1 | 23 | + |
| 71 | guide | sgRNA for R126L (for | R126 | n.a. | n.a. | n.a. | n.a. | n.a. | n.a. | n.a. | n.a. | n.a. | Cas9 | 20 | + |
| | | Cas9) | | | | | | | | | | | | | |
| 72 | guide | sgRNA for G398A (for | G398 | n.a. | n.a. | n.a. | n.a. | n.a. | n.a. | n.a. | n.a. | n.a. | Cpf1 | 20 | + |
| | | Cpf1) | | | | | | | | | | | | | |
| 73 | guide | sgRNA for L397E and | L397 | F420 | n.a. | n.a. | n.a. | n.a. | n.a. | n.a. | n.a. | n.a. | Cpf1 | 20 | + |
| | | F420V (for Cpf1) | | | | | | | | | | | | | |
| 74 | guide | sgRNA2 for R126 | R126 | n.a. | n.a. | n.a. | n.a. | n.a. | n.a. | n.a. | n.a. | n.a. | Cpf1 | 23 | + |
| 75 | guide | sgRNA1 for G208 | G208/ | n.a. | n.a. | n.a. | n.a. | n.a. | n.a. | n.a. | n.a. | n.a. | Cpf1 | 23 | + |
| | | | G209 | | | | | | | | | | | | |
| 76 | guide | sgRNA2 for G208 | G208/ | n.a. | n.a. | n.a. | n.a. | n.a. | n.a. | n.a. | n.a. | n.a. | Cpf1 | 23 | + |
| | | | G209 | | | | | | | | | | | | |
| 77 | guide | sgRNA1 for F420 | F420 | n.a. | n.a. | n.a. | n.a. | n.a. | n.a. | n.a. | n.a. | n.a. | Cpf1 | 23 | + |
| 78 | guide | sgRNA2 for F420 | F420 | n.a. | n.a. | n.a. | n.a. | n.a. | n.a. | n.a. | n.a. | n.a. | Cpf1 | 23 | + |
| 79 | guide | sgRNA for G208/209 (for | G208/ | n.a. | n.a. | n.a. | n.a. | n.a. | n.a. | n.a. | n.a. | n.a. | Cas9 | 20 | − |
| | | Cas9) | G209 | | | | | | | | | | | | |
| 80 | guide | sgRNA for G398A (for | G398 | n.a. | n.a. | n.a. | n.a. | n.a. | n.a. | n.a. | n.a. | n.a. | Cas9 | 20 | − |
| | | Cas9) | | | | | | | | | | | | | |
| 81 | primer | KASP marker primer | R126 | n.a. | n.a. | n.a. | n.a. | n.a. | n.a. | n.a. | n.a. | n.a. | n.a. | 41 | |
| | | alleleX S01/S02 | | | | | | | | | | | | | |
| 82 | primer | KASP marker primer | R126 | n.a. | n.a. | n.a. | n.a. | n.a. | n.a. | n.a. | n.a. | n.a. | n.a. | 47 | |
| | | alleleX S03 | | | | | | | | | | | | | |
| 83 | primer | KASP marker primer | R126 | n.a. | n.a. | n.a. | n.a. | n.a. | n.a. | n.a. | n.a. | n.a. | n.a. | 43 | |
| | | alleleY S01/S02 | | | | | | | | | | | | | |
| 84 | primer | KASP marker primer | R126 | n.a. | n.a. | n.a. | n.a. | n.a. | n.a. | n.a. | n.a. | n.a. | n.a. | 49 | |
| | | alleleY S03 | | | | | | | | | | | | | |
| 85 | primer | Sanger forward primer | R126 | n.a. | n.a. | n.a. | n.a. | n.a. | n.a. | n.a. | n.a. | n.a. | n.a. | 29 | |
| 86 | primer | Sanger reverse primer | R126 | n.a. | n.a. | n.a. | n.a. | n.a. | n.a. | n.a. | n.a. | n.a. | n.a. | 28 | |
| 87 | primer | KASP marker primer | R126 | n.a. | n.a. | n.a. | n.a. | n.a. | n.a. | n.a. | n.a. | n.a. | n.a. | 29 | |
| | | common S01 | | | | | | | | | | | | | |
| 88 | primer | KASP marker primer | R126 | n.a. | n.a. | n.a. | n.a. | n.a. | n.a. | n.a. | n.a. | n.a. | n.a. | 30 | |
| | | common S02 | | | | | | | | | | | | | |
| 89 | primer | KASP marker primer | R126 | n.a. | n.a. | n.a. | n.a. | n.a. | n.a. | n.a. | n.a. | n.a. | n.a. | 25 | |
| | | common S03 | | | | | | | | | | | | | |
| 90 | donor | Donor template for G208/209 | G208/ | n.a. | GGA | n.a. | — | n.a. | — | n.a. | CAGTCT* | CAaTCc* | Cas9 | 175 | + |
| | | with silent mutation (for | G209 | | | | | | | | | | | | |
| | | Cas9) | | | | | | | | | | | | | |
| 91 | donor | Donor template for | G398 | n.a. | GGT | n.a. | GCN | n.a. | A | n.a. | GTTACGAAATCAGGG | aTTgCGgAATCAGcG | Cas9 | 155 | − |
| | | G398A with silent | | | | | | | | | | | | | |
| | | mutation (for Cas9) | | | | | | | | | | | | | |
| 92 | donor | Donor template L397E | L397 | F420 | CTC | TTT | GAR | GTN | E | V | AATTGTCTGGTT . . . GTTGGAGGA | AAcTGcCTcGTT . . . GTcGGcGGA | Cas9 | 200 | + |
| | | and F420V with silent | | | | | | | | | | | | | |
| | | mutation (for Cas9) | | | | | | | | | | | | | |
| 93 | donor | Donor template for | R126 | n.a. | AGA | n.a. | TTR | n.a. | L | n.a. | ATTGCACAA | ATcGCgCAg | Cpf1 | 161 | + |
| | | R126L with silent | | | | | | | | | | | | | |
| | | mutation (for Cpf1) | | | | | | | | | | | | | |
| 94 | donor | Donor template for G208/209 | G208/ | n.a. | GGA | n.a. | — | n.a. | — | n.a. | GACAAAAGG | cACgAAgGG | Cpf1 | 154 | − |
| | | with silent mutation (for | G209 | | | | | | | | | | | | |
| | | Cpf1) | | | | | | | | | | | | | |
| 95 | donor | Donor template for | G398 | n.a. | GGT | n.a. | GCN | n.a. | A | n.a. | CAGGGTTTGAAAA | CAaGGaTTGAgAA | Cpf1 | 155 | − |
| | | G398A with silent | | | | | | | | | | | | | |
| | | mutation (for Cpf1) | | | | | | | | | | | | | |
| 96 | donor | Donor template for | F420 | n.a. | TTT | n.a. | GTN | n.a. | V | n.a. | ATCGGAGAAAGC | gTCtGAcAAtGC | Cpf1 | 191 | − |
| | | F420V with silent | | | | | | | | | | | | | |
| | | mutation (for Cpf1) | | | | | | | | | | | | | |
| 97 | gDNA | Genomic DNA (gDNA) | G209 | n.a. | GGA | n.a. | — | n.a. | — | n.a. | n.a. | n.a. | Cas9 | 37006 | + |
| | | sequence of G208/209 mutant | | | | | | | | | | | | | |
| | | of Beta vulgaris PPO2 | | | | | | | | | | | | | |
| | | with silent mutation | | | | | | | | | | | | | |
| 98 | CDS | cDNA sequence of G208/209 | G209 | n.a. | GGA | n.a. | — | n.a. | — | n.a. | n.a. | n.a. | Cas9 | 1572 | |
| | | mutant of Beta vulgaris | | | | | | | | | | | | | |
| | | PPO2 | | | | | | | | | | | | | |
| 99 | gDNA | Genomic DNA (gDNA) | G398 | n.a. | GGT | n.a. | GCT | n.a. | A | n.a. | n.a. | n.a. | Cas9 | 37009 | |
| | | sequence of G398A | | | | | | | | | | | | | |
| | | mutant of Beta vulgaris | | | | | | | | | | | | | |
| | | PPO2 with silent mutation | | | | | | | | | | | | | |
| 100 | gDNA | Genomic DNA (gDNA) | L397 | F420 | CTC | TTT | GAR | GTN | E | V | n.a. | n.a. | Cas9 | 37009 | |
| | | sequence of L397E and | | | | | | | | | | | | | |
| | | F420V mutant of Beta | | | | | | | | | | | | | |
| | | vulgaris PPO2 with silent | | | | | | | | | | | | | |
| | | mutation | | | | | | | | | | | | | |
| 101 | CDS | cDNA sequence of L397E | L397 | F420 | CTC | TTT | GAR | GTN | E | V | n.a. | n.a. | Cas9 | 1575 | |
| | | and F420V mutant of | | | | | | | | | | | | | |
| | | Beta vulgaris PPO2 | | | | | | | | | | | | | |
| 102 | gDNA | Genomic DNA (gDNA) | R126 | n.a. | AGA | n.a. | CTN | n.a. | L | n.a. | n.a. | n.a. | Cpf1 | 37009 | |
| | | sequence of R126L | | | | | | | | | | | | | |
| | | mutant of Beta vulgaris | | | | | | | | | | | | | |
| | | PPO2 with silent mutation | | | | | | | | | | | | | |
| 103 | CDS | cDNA sequence of | R126 | n.a. | AGA | n.a. | CTN | n.a. | L | n.a. | n.a. | n.a. | Cpf1 | 1575 | |
| | | R126L mutant of Beta | | | | | | | | | | | | | |
| | | vulgaris PPO2 | | | | | | | | | | | | | |
| 104 | gDNA | Genomic DNA (gDNA) | G209 | n.a. | GGA | n.a. | — | n.a. | — | n.a. | n.a. | n.a. | Cpf1 | 37006 | |
| | | sequence of G208/209 mutant | | | | | | | | | | | | | |
| | | of Beta vulgaris PPO2 | | | | | | | | | | | | | |
| | | with silent mutation | | | | | | | | | | | | | |
| 105 | CDS | cDNA sequence of G208/209 | G209 | n.a. | GGA | n.a. | — | n.a. | — | n.a. | n.a. | n.a. | Cpf1 | 1572 | |
| | | mutant of Beta vulgaris | | | | | | | | | | | | | |
| | | PPO2 | | | | | | | | | | | | | |
| 106 | gDNA | Genomic DNA (gDNA) | G398 | n.a. | GGT | n.a. | GCT | n.a. | A | n.a. | n.a. | n.a. | Cpf1 | 37009 | |
| | | sequence of G398A | | | | | | | | | | | | | |
| | | mutant of Beta vulgaris | | | | | | | | | | | | | |
| | | PPO2 with silent mutation | | | | | | | | | | | | | |
| 107 | gDNA | Genomic DNA (gDNA) | L397 | F420 | CTC | TTT | GAR | GTN | E | V | n.a. | n.a. | Cpf1 | 37009 | |
| | | sequence of F420V | | | | | | | | | | | | | |
| | | mutant of Beta vulgaris | | | | | | | | | | | | | |
| | | PPO2 with silent mutation | | | | | | | | | | | | | |
| 108 | CDS | cDNA sequence of L397E | L397 | F420 | CTC | TTT | GAR | GTN | E | V | n.a. | n.a. | Cpf1 | 1575 | |
| | | and F420V mutant of | | | | | | | | | | | | | |
| | | Beta vulgaris PPO2 | | | | | | | | | | | | | |
| 109 | primer | KASP marker primer | R126 | n.a. | n.a. | n.a. | n.a. | n.a. | n.a. | n.a. | n.a. | n.a. | n.a. | 45 | |
| | | alleleX S04 | | | | | | | | | | | | | |
| 110 | primer | KASP marker primer | R126 | n.a. | n.a. | n.a. | n.a. | n.a. | n.a. | n.a. | n.a. | n.a. | n.a. | 48 | |
| | | alleleY S04 | | | | | | | | | | | | | |
| 111 | primer | KASP marker primer | R126 | n.a. | n.a. | n.a. | n.a. | n.a. | n.a. | n.a. | n.a. | n.a. | n.a. | 26 | |
| | | common S04 | | | | | | | | | | | | | |
| 112 | primer | KASP marker allele | G208/ | n.a. | n.a. | n.a. | n.a. | n.a. | n.a. | n.a. | n.a. | n.a. | n.a. | 45 | |
| | | S01/S02 | 209 | | | | | | | | | | | | |
| 113 | primer | KASP marker allele | G208/ | n.a. | n.a. | n.a. | n.a. | n.a. | n.a. | n.a. | n.a. | n.a. | n.a. | 45 | |
| | | S01/S02/S04 | 209 | | | | | | | | | | | | |
| 114 | primer | KASP marker primer | G208/ | n.a. | n.a. | n.a. | n.a. | n.a. | n.a. | n.a. | n.a. | n.a. | n.a. | 29 | |
| | | common S01 | 209 | | | | | | | | | | | | |
| 115 | primer | KASP marker primer | G208/ | n.a. | n.a. | n.a. | n.a. | n.a. | n.a. | n.a. | n.a. | n.a. | n.a. | 30 | |
| | | common S02/S04 | 209 | | | | | | | | | | | | |
| 116 | primer | KASP marker allele S03 | G208/ | n.a. | n.a. | n.a. | n.a. | n.a. | n.a. | n.a. | n.a. | n.a. | n.a. | 42 | |
| | | | 209 | | | | | | | | | | | | |
| 117 | primer | KASP marker alleleY | G208/ | n.a. | n.a. | n.a. | n.a. | n.a. | n.a. | n.a. | n.a. | n.a. | n.a. | 41 | |
| | | S03/A05 | 209 | | | | | | | | | | | | |
| 118 | primer | KASP marker primer | G208/ | n.a. | n.a. | n.a. | n.a. | n.a. | n.a. | n.a. | n.a. | n.a. | n.a. | 30 | |
| | | common S03/S05 | 209 | | | | | | | | | | | | |
| 119 | primer | KASP marker allele S04 | G208/ | n.a. | n.a. | n.a. | n.a. | n.a. | n.a. | n.a. | n.a. | n.a. | n.a. | 45 | |
| | | | 209 | | | | | | | | | | | | |
| 120 | primer | KASP marker allele S05 | G208/ | n.a. | n.a. | n.a. | n.a. | n.a. | n.a. | n.a. | n.a. | n.a. | n.a. | 42 | |
| | | | 209 | | | | | | | | | | | | |
| 121 | primer | Sanger forward primer | G208/ | n.a. | n.a. | n.a. | n.a. | n.a. | n.a. | n.a. | n.a. | n.a. | n.a. | 31 | |
| | | | 209 | | | | | | | | | | | | |
| 122 | primer | Sanger reverse primer | G208/ | n.a. | n.a. | n.a. | n.a. | n.a. | n.a. | n.a. | n.a. | n.a. | n.a. | 30 | |
| | | | 209 | | | | | | | | | | | | |

OC1—original codon 1
OC2—original codon 2
CC1—codon change 1
CC2—codon change 2
AA1—amino acid 1
AA2—amino acid 2
Sil Mut OS—Original sequence
Sil Mut CS—Changed sequence after introducing silent mutations (donor template)
+/− strand
*For SEQ ID: 90, the silent mutation in the seed region of the guide, not in the PAM site

### DETAILED DESCRIPTION

### Definitions

While the following terms are believed to be well understood by one of ordinary skill in the art, the following definitions are set forth to facilitate explanation of the presently disclosed subject matter.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD).

In some embodiments, one or more of the elements to which the “and/or” refers can also individually be present in single or multiple occurrences in the combinations(s) and/or subcombination(s).

The term “engineered” or “genetically engineered” refers to any man-made manipulation of a genome of a cell of interest (e.g., by insertion, deletion or substitution of nucleic acids). In some embodiments the term “engineered” means that (i) at least one of the genetic changes to the nucleic acid encoding a protoporphyrinogen oxidase 2 (PPO2) amino acid sequence is not exclusively obtained by an essentially biological process or (ii) said nucleic acid encoding a protoporphyrinogen oxidase 2 (PPO2) amino acid sequence has been introduced or modified by a step of a technical nature so that the introduction or modification is not exclusively the result of the mixing of the genes of the plants by sexual crossing.

“Homologous sequences” or “homologs” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as NCBI BLAST (Basic Local Alignment Search Tool), using default parameters.

As used herein, the term “at least a portion” or “fragment” of a nucleic acid or polypeptide means a portion having the minimal size characteristics of such sequences, or any larger fragment of the full-length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full-length polypeptide. The length of the portion to be used will depend on the particular application. A portion of a nucleic acid useful as a hybridization probe or targeting region of a guide RNA may be as short as 12 nucleotides; in some aspects, it is or is about 15, 20, or 25 nucleotides. A portion of a polypeptide useful as an epitope may be as short as 4 amino acids. A portion of a polypeptide that performs the function of the full-length polypeptide would generally be longer than 4 amino acids. In some cases, a portion of a polypeptide that performs the function of the full-length polypeptide contains 1, 2, 3, 4, 5, 6, 7, 8, 9, or 10 amino acids deleted from the N and/or C-terminus.

As used herein, the terms “endogenous,” and “native” refer to the naturally occurring copy of a gene or promoter.

As used herein, the term “naturally occurring” refers to a gene derived from a naturally occurring source. In some aspects, a naturally occurring gene refers to a gene of a wild type (non-transgene) gene, whether located in its endogenous setting within the source organism, or if placed in a “heterologous” setting, when introduced in a different organism. Thus, for the purposes of this disclosure, a “non-naturally occurring” gene is a gene that has been mutated or otherwise modified, or synthesized, to have a different sequence from known natural genes. In some aspects, the modification may be at the protein level (e.g., amino acid substitutions). In other aspects, the modification may be at the DNA level, without any effect on protein sequence (e.g., codon optimization).

As used herein, the term “heterologous” refers to an amino acid or a nucleic acid sequence (e.g., gene or promoter), which is not naturally found in the particular organism or is not naturally found in a particular context (e.g., genomic or plasmid location) in the particular organism. For example, a native promoter or other nucleic acid sequence of Beta vulgaris can be heterologous when operably linked to a nucleic acid sequence it is not operably linked to in a wild-type Beta vulgaris, or when it is delivered in a non-native form such as in a heterologous plasmid or a heterologous nucleic acid fragment.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “transgenic” refers to an organism that contains genetic material into which DNA from another species has been artificially introduced. The term “non-transgenic” thus refers to an organism which does not comprise genetic material from another species.

As used herein, the term “cisgenesis” refers to genetic modification of a recipient organism with a gene (cisgene) from a crossable, sexually compatible, organism.

As used herein, the term “intragenesis” is genetic modification of a recipient organism that involves the insertion of a reorganized, full or partial coding region of a gene combined frequently with a promoter and/or terminator from another gene of the same species or a crossable species.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746).

The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern blot analysis of DNA, Northern blot analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

The term “operably linked” means in this context the sequential arrangement of the promoter polynucleotide according to the disclosure with a further oligo- or polynucleotide, resulting in transcription of said further polynucleotide. In some aspects, the promoter sequences of the present disclosure are inserted just prior to a gene's 5′UTR, or open reading frame. In other aspects, the operably linked promoter sequences and gene sequences of the present disclosure are separated by one or more linker nucleotides.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “target nucleic acid” as used herein is a polynucleotide (e.g., RNA, DNA) that includes a “target site” or “target sequence.” The terms “target site” or “target sequence” are used interchangeably herein to refer to a nucleic acid sequence present in a target nucleic acid to which a targeting segment of a subject guide nucleic acid will bind, provided sufficient conditions for binding exist. Suitable hybridization conditions include physiological conditions normally present in a cell. For a double stranded target nucleic acid, the strand of the target nucleic acid that is complementary to and hybridizes with the guide nucleic acid is referred to as the “complementary strand”; while the strand of the target nucleic acid that is complementary to the “complementary strand” (and is therefore not complementary to the guide nucleic acid) is referred to as the “noncomplementary strand” or “non-complementary strand”. In embodiments where the target nucleic acid is a single stranded target nucleic acid (e.g., single stranded DNA (ssDNA), single stranded RNA (ssRNA)), the guide nucleic acid is complementary to and hybridizes with single stranded target nucleic acid.

A nucleic acid molecule that binds to an RNA-guided endonuclease (e.g., the Cas9 Polypeptide) and targets the polypeptide to a specific location within the target nucleic acid is referred to herein as a “guide nucleic acid”. When the guide nucleic acid is an RNA molecule, it can be referred to as a “guide RNA” or a “gRNA”. A guide nucleic acid comprises two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. For example, in some embodiments the protein-binding segment (described below) of a guide nucleic acid is one nucleic acid molecule (e.g., one RNA molecule) and the protein-binding segment therefore comprises a region of that one molecule. In other embodiments, the protein-binding segment (described below) of a guide nucleic acid comprises two separate molecules that are hybridized along a region of complementarity. As an illustrative, non-limiting example, a protein-binding segment of a guide nucleic acid that comprises two separate molecules can comprise (i) base pairs 40-75 of a first molecule (e.g., RNA molecule, DNA/RNA hybrid molecule) that is 100 base pairs in length; and (ii) base pairs 10-25 of a second molecule (e.g., RNA molecule) that is 50 base pairs in length. The definition of “segment,” unless otherwise specifically defined in a particular context, is not limited to a specific number of total base pairs, is not limited to any particular number of base pairs from a given nucleic acid molecule, is not limited to a particular number of separate molecules within a complex, and may include regions of nucleic acid molecules that are of any total length and may or may not include regions with complementarity to other molecules.

The first segment (targeting segment) of a guide nucleic acid (e.g., guide RNA or gRNA) comprises a nucleotide sequence that is complementary to a specific sequence (a target site) within a target nucleic acid (e.g., a target ssRNA, a target ssDNA, the complementary strand of a double stranded target DNA, etc.). The protein-binding segment (or “protein-binding sequence”) interacts with an RNA-guided endonuclease (e.g., Cas9) polypeptide. Site-specific binding and/or cleavage of the target nucleic acid can occur at locations determined by base-pairing complementarity between the guide nucleic acid (e.g., guide RNA) and the target nucleic acid.

The protein-binding segment of a subject guide nucleic acid comprises two complementary stretches of nucleotides that hybridize to one another to form a double stranded RNA duplex (dsRNA duplex).

A subject guide nucleic acid (e.g., guide RNA) linked to a donor polynucleotide forms a complex with a subject RNA-guided endonuclease (e.g., Cas9) (i.e., binds via non-covalent interactions). The guide nucleic acid (e.g., guide RNA) provides target specificity to the complex by comprising a nucleotide sequence that is complementary to a sequence of a target nucleic acid. Thus, the RNA-guided endonuclease (e.g., Cas9) of the complex provides site-specific or “targeted” activity by virtue of its association with the protein-binding segment of the guide nucleic acid.

In some embodiments, a subject guide nucleic acid (e.g., guide RNA) comprises two separate nucleic acid molecules and is referred to herein as a “dual guide nucleic acid.” In some embodiments, the subject guide nucleic acid is a single nucleic acid molecule (single polynucleotide) and is referred to herein as a “single guide nucleic acid.” The term “guide nucleic acid” is inclusive, referring to both dual guide nucleic acids and to single guide nucleic acids and the term “guide RNA” is also inclusive, referring to both dual guide RNA (dgRNA) and single guide RNA (sgRNA).

In some embodiments, a guide nucleic acid is a DNA/RNA hybrid molecule. In such embodiments, the protein-binding segment of the guide nucleic acid is RNA and forms an RNA duplex. However, the targeting segment of a guide nucleic acid can be DNA. Thus, if a DNA/RNA hybrid guide nucleic acid is a dual guide nucleic acid, the targeting segment can be DNA and the duplex-forming segment can be RNA. In such embodiments, the duplex-forming segment of the “activator” molecule can be RNA (e.g., in order to form an RNA-duplex with the duplex-forming segment of the targeting segment), while nucleotides of the “activator” molecule that are outside of the duplex-forming segment can be DNA (in which case the activator molecule is a hybrid DNA/RNA molecule) or can be RNA (in which case the activator molecule is RNA). If a DNA/RNA hybrid guide nucleic acid is a single guide nucleic acid, then the targeting segment can be DNA, the duplex-forming segments (which make up the protein-binding segment) can be RNA, and nucleotides outside of the targeting and duplex-forming segments can be RNA or DNA.

An exemplary dual guide nucleic acid comprises a CRISPR-RNA (crRNA) molecule and a corresponding trans-activating crRNA (tracrRNA) molecule. The crRNA molecule comprises both the targeting segment (single stranded) of the guide nucleic acid and a stretch (“duplex-forming segment”) of nucleotides that forms one half of the dsRNA duplex of the protein-binding segment of the guide nucleic acid. The corresponding tracrRNA molecule comprises a stretch of nucleotides (duplex-forming segment) that forms the other half of the dsRNA duplex of the protein-binding segment of the guide nucleic acid. In other words, a stretch of nucleotides of a crRNA molecule are complementary to and hybridize with a stretch of nucleotides of a tracrRNA molecule to form the dsRNA duplex of the protein-binding domain of the guide nucleic acid. The crRNA-like molecule additionally provides the single stranded targeting segment. Thus, the crRNA and the tracrRNA (as a corresponding pair) hybridize to form a dual guide nucleic acid. The exact sequence of a given crRNA or tracrRNA molecule is characteristic of the species in which the RNA molecules are found.

The term “protospacer” refers to the DNA sequence targeted by a crRNA guide strand.

In some aspects the protospacer sequence hybridizes with the crRNA guide sequence of a CRISPR complex.

The “protospacer-adjacent motif” or “PAM” sequence is a 2-6 base pair DNA sequence immediately following the DNA sequence targeted by an RNA-guided endonuclease (e.g., Cas9). The PAM sequences is required for cleavage of the target nucleic acid and varies depending on the source of the RNA-guided endonuclease (e.g., Cas9). For example, in case of the Streptococcus pyogenes Cas9 the PAM sequence is NGG. In aspects of the present disclosure, the PAM sequences is mutated by the donor polynucleotide such that further cleavage of the target site is prevented. If it is not possible to introduce silent mutations in the PAM sequence, these can also be introduced in the seed region of the gRNA, as in SEQ ID NO: 90.

In some instances, a component, e.g., a nucleic acid component (e.g., a guide nucleic acid, etc.); a protein component (e.g., an RNA-guided endonuclease, a Cas9 polypeptide, a variant RNA-guided endonuclease, a variant Cas9 polypeptide); and the like) includes a label moiety. The terms “label”, “detectable label”, or “label moiety” as used herein refer to any moiety that provides for signal detection and may vary widely depending on the particular nature of the assay. Label moieties of interest include both directly detectable labels (e.g., a fluorescent label) and indirectly detectable labels (indirect labels, e.g., a binding pair member). A fluorescent label can be any fluorescent label, e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), mCherry, mTomato, mTangerine, and any fluorescent derivative thereof, etc.).

Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label) (e.g., 3H, 125I, 35S, 14C, or 32P); an enzyme (an indirect label) (e.g., peroxidase, alkaline phosphatase, galactosidase, luciferase, glucose oxidase, and the like); a fluorescent protein (a direct label) (e.g., green fluorescent protein, red fluorescent protein, yellow fluorescent protein, and any convenient derivatives thereof); a metal label (a direct label); a colorimetric label; a binding pair member; and the like. By “binding pair member” is meant one of a first and a second moiety, wherein the first and the second moiety have a specific binding affinity for each other.

Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Sequence identity. “sequence identity” or “identity” in the context of two nucleic acid or polypeptide sequences includes reference to the number of residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). The comparison of sequences and determination of percent identity between two sequences can be accomplished using a mathematical algorithm. An example of a local alignment algorithm utilized for the comparison of sequences is the NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)'s world-wide-web URL. A description of how to determine sequence identity using this program is available at the NLM's website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal Omega. Unless otherwise stated, references to sequence identity used herein refer to the Clustal Omega.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores.

The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “resistant”, or “resistance”, describes a plant, line or variety that shows fewer or reduced symptoms to a herbicide than a susceptible (or more susceptible) plant, line or variety to that herbicide. This term is also applied to plants that show no symptoms, and may also be referred to as “high/standard resistance”.

As used herein, the term “tolerant” or “tolerance” describes a plant, line, or variety that that shows some symptoms to a herbicide, but that are still able to produce marketable product with an acceptable yield. These lines may also be referred to as having “moderate/intermediate resistance”. Tolerant and moderate/intermediate resistant plant types are affected by a herbicide with a greater range of symptoms or damage compared to plant types with high resistance.

Plant types with intermediate resistance will show less severe symptoms than susceptible plant varieties, when grown under similar field conditions and herbicide treatment.

Methods of evaluating resistance are well known to one skilled in the art. Such evaluation may be performed by visual observation of a plant or a plant part (e.g., leaves, roots, flowers, fruits et. al) in determining the severity of symptoms. For example, when each plant is given a resistance score on a scale of 1 to 5 based on the severity of the reaction or symptoms, with 1 being the resistance score applied to the most resistant plants (e.g., no symptoms, or with the least symptoms), and 5 the score applied to the plants with the most severe symptoms, then a line is rated as being resistant when at least 75% of the plants have a resistance score at a 1, 2, or 3 level, while susceptible lines are those having more than 25% of the plants scoring at a 4 or 5 level. In some embodiments, at least 90% of the plants in a resistant line will have a score of 1, 2, or 3. If a more detailed visual evaluation is possible, then one can use a scale from 1 to 10 so as to broaden out the range of scores and thereby hopefully provide a greater scoring spread among the plants being evaluated. Instead of scoring individual plants, one can also provide a score on a group of plants, where the plants in one group would belong to the same line and be clones of each other. Any given component, or combination of components can be unlabeled, or can be detectably labeled with a label moiety. In some embodiments, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); Cell and Tissue Culture: Laboratory Procedures in Biotechnology (Doyle & Griffiths, John Wiley & Sons 1998); and Current Protocols in Molecular Biology (Ausubel et al. eds., John Wiley & Sons 2003), including supplements 1-117, the disclosures of which are incorporated herein by reference.

### Overview

The present disclosure relates to Beta vulgaris plants having resistance to PPO herbicides and methods of producing said plants by targeted genome editing. The disclosure further relates to genetic sequences for use with targeted genome editing technologies and/or genotyping, and herbicide-resistant PPO proteins produced from genetically engineered, non-transgenic Beta vulgaris plants.

Agricultural crop production often utilizes herbicide tolerance (HT) traits predominantly introduced by conventional plant transformation methods, which results in transgenic crops with the desired traits. However, DNA can be modified in a targeted way using genome editing techniques, to develop novel, desired traits in plants of interest. The present disclosure teaches utilization of an emerging technology including the targeted genome editing techniques, such as CRISPR/Cas system, to establish herbicide tolerance traits, thereby producing non-transgenic crops with the desired traits.

The disclosure provides introduction of commercially relevant herbicide tolerance traits into crops of interest by editing the endogenous PPO genes in a targeted, non-transgenic manner. Thus, the disclosure provides genetically engineered, endogenous herbicide-tolerant protoporphyrinogen oxidases (PPO) useful for providing PPO herbicide tolerance in the crops of interest, including fodder beet and sugar beet.

Also, the disclosure provides making a non-transgenic plant with the herbicide tolerance traits introduced by the genome editing technique taught herein, and further producing the non-transgenic plant combined with one or more other herbicide-tolerance trait(s).

Beta vulgaris

Beta vulgaris (“Beet”), is a root vegetable of the subfamily Betoideae within the family Amaranthaceae. Examples of beet include sugar beet, garden beets (red beet), leafy beets (chard), and fodder beets (forage). Sugar beet (B. vulgaris L. ssp. vulgaris) is grown both as a garden vegetable and, since the mid-18^{th} century, for its sugar content. Sugar from sugar beet accounts for approximately 20-30% of the world's annual production of sugar, the rest being extracted from sugar cane (Yamane, Takeo. “Sugar beet”. Encyclopedia Britannica, 12 Apr. 2016, available on the world wide web at britannica.com/plant/sugar-beet; Dohm, J., Minoche, A., Holtgräwe, D. et al. The genome of the recently domesticated crop plant sugar beet (Beta vulgaris) is published in Nature 505, 546-549 (2014)).

As sugar beet has reached such a high yield potential, new tolerances and resistances are needed for herbicides such as PPO herbicides.

### Protoporphyrinogen Oxidase (PPO) Herbicides

As used herein, “herbicide” is any molecule that is used to control, prevent, or interfere with the growth of one or more undesired plants in a cultivated area (e.g. weeds). Exemplary herbicides include acetyl-CoA carboxylase (ACCase) inhibitors (for example aryloxyphenoxy propionates and cyclohexanediones); acetolactate synthase (ALS) inhibitors (for example sulfonylureas, imidazolinones, triazolopyrimidines, and triazolinones); 5-enolpyruvylshikimate-3-phosphate synthase (EPSPS) inhibitors (for example glyphosate), synthetic auxins (for example phenoxys, benzoic acids, carboxylic acids, semicarbazones), photosynthesis (photosystem II) inhibitors (for example triazines, triazinones, nitriles, benzothiadiazoles, and ureas), glutamine synthetase (GS) inhibitors (for example glufosinate and bialaphos), 4-hydroxyphenylpyruvate dioxygenase (HPPD) inhibitors (for example isoxazoles, pyrazolones, and triketones), protoporphyrinogen oxidase (PPO) inhibitors (for example diphenylethers, N-phenylphthalimide, aryl triazinones, and pyrimidinediones), very long-chain fatty acid inhibitors (for example chloroacetamides, oxyacetamides, and pyrazoles), cellulose biosynthesis inhibitors (for example indaziflam), photosystem I inhibitors (for example paraquat), microtubule assembly inhibitors (for example pendimethalin), and phytoene desaturase (PDS) inhibitors (for example norflurazone), among others.

A PPO herbicide is a chemical that targets and inhibits the enzymatic activity of a protoporphyrinogen oxidase (PPO), which catalyzes the dehydrogenation of protoporphyrinogen IX to form protoporphyrin IX, which is the precursor to heme and chlorophyll. Inhibition of protoporphyrinogen oxidase causes formation of reactive oxygen species, resulting in cell membrane disruption and ultimately the death of susceptible cells. PPO herbicides are well-known in the art and commercially available. Exemplary PPO herbicides are shown in Table 2 below.

TABLE 2
Exemplary PPO herbicides

| PPO Herbicide | Chemical Family | 1X Rate | |
|---|---|---|---|
| acifluorfen | Diphenylethers | 420 | g ai/ha |
| fomesafen | Diphenylethers | 420 | g ai/ha |
| lactofen | Diphenylethers | 70-220 | g ai/ha |
| fluoroglycofen-ethyl | Diphenylethers | 15-40 | g ai/ha |
| oxyfluorfen | Diphenylethers | 0.28-2.24 | kg ai/ha |
| flumioxazin | N-phenylphthalimide | 70-105 | g ai/ha |
| azafenidin | Triazolinone | 240 | g ai/ha |
| casfentrazone-ethyl | Triazolinone | 4-36 | g ai/ha |
| sulfentrazone | Triazolinone | 0.1-0.42 | kg ai/ha |
| fluthiacet-methyl | Thiadiazole | 3-15 | g ai/ha |
| oxadiargyl | Oxadiazole | 50-150 | g ai/ha |
| oxadiazon | Oxadiazole | 2.24-4.48 | kg ai/ha |
| pyraflufen-ethyl | Phenylpyrazole | 6-12 | g ai/ha |
| saflufenzeil | Pyrimidine dione | 23-50 | g/ha |
| S-3100 | Pyrimidine dione | 5-80 | g/ha |

Examples of PPO herbicides include, but are not limited to diphenylethers (such as acifluorfen, its salts and esters, aclonifen, bifenox, its salts and esters, ethoxyfen, its salts and esters, fluoronitrofen, furyloxyfen, halosafen, chlomethoxyfen, fluoroglycofen, its salts and esters, lactofen, its salts and esters, oxyfluorfen, and fomesafen, its salts and esters); thiadiazoles (such as fluthiacet-methyl and thidiazimin); pyrimidinediones or phenyluracils (such as benzfendizone, butafenacil, ethyl [3-2-chloro-4-fluoro-5-(d-methyl-6-trifluoromethyl-2,4-dioxo-1,2,3,4-tetrahydropyrimidin-3-yl)phenoxy]-2-pyridyloxy] acetate (CAS Registry Number 353292-31-6 and referred to herein as S-3100), trifludimoxazin, flupropacil, saflufenacil, and tiafenacil); phenylpyrazoles (such as fluazolate, pyraflufen and pyraflufen-ethyl); oxadiazoles (such as oxadiargyl and oxadiazon); triazolinones (such as azafenidin, bencarbazone, carfentrazone, its salts and esters, and sulfentrazone); oxazolidinediones (such as pentoxazone); N-phenylphthalimides (such as cinidon-ethyl, flumiclorac, flumiclorac-pentyl, and flumioxazin); benzoxazinone derivatives (such as 1,5-dimethyl-6-thioxo-3-(2,2,7-triflworo-3,4-dihydro-3-oxo-4-prop-2-ynyl-2//-1,4-benzoxazin-6-yl)-1,3,5-triazinane-2,4-dione); flufenpyr and flufenpyr-ethyl; pyraclonil; trifludimoxazin, and profluazol.

### Targeted Genome Editing for Production of Engineered Proteins

The genome of sugar beet was recently sequenced (Dohm, J., Minoche, A., Holtgräwe, D. et al. The genome of the recently domesticated crop plant sugar beet (Beta vulgaris). Nature 505, 546-549 (2014)). Emerging genome editing technology provides the opportunity to establish non-transgenic herbicide tolerant traits, which substantially reduces costs and development times, which is key for crops like sugar beet which have a limited market size. In other crops it has previously been reported that PPO herbicide tolerance was only achievable through overexpression of resistant genes (Lermontova I, Grimm B. Overexpression of plastidic protoporphyrinogen IX oxidase leads to resistance to the diphenyl-ether herbicide acifluorfen. Plant Physiology. 2000; 122(1):75-84; Adhikari P, Goodrich F, Fernandes S B, et al. Genetic variation associated with PPO-inhibiting herbicide tolerance in sorghum. PLoS One. 2020; 15(10) Published 2020 Oct. 14; Li X, Nicholl D. Development of PPO inhibitor-resistant cultures and crops. Pest Manag Sci. 2005 March; 61(3):277-85). However, as disclosed here the inventors found that commercially relevant herbicide tolerance in sugar beet was achieved by editing the endogenous PPO genes.

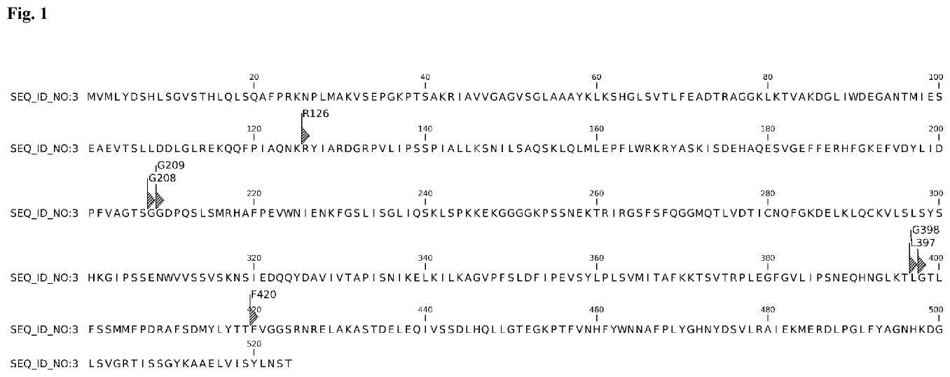

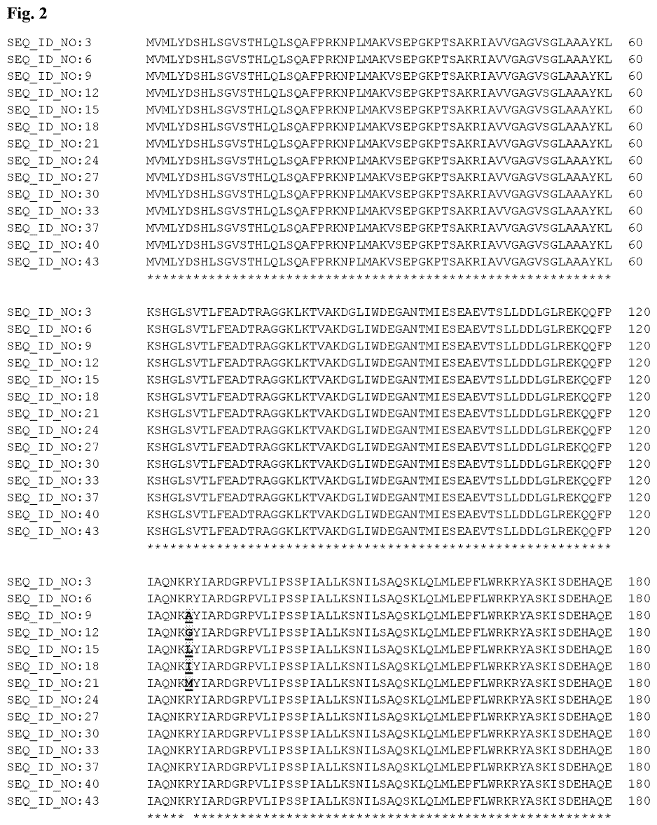

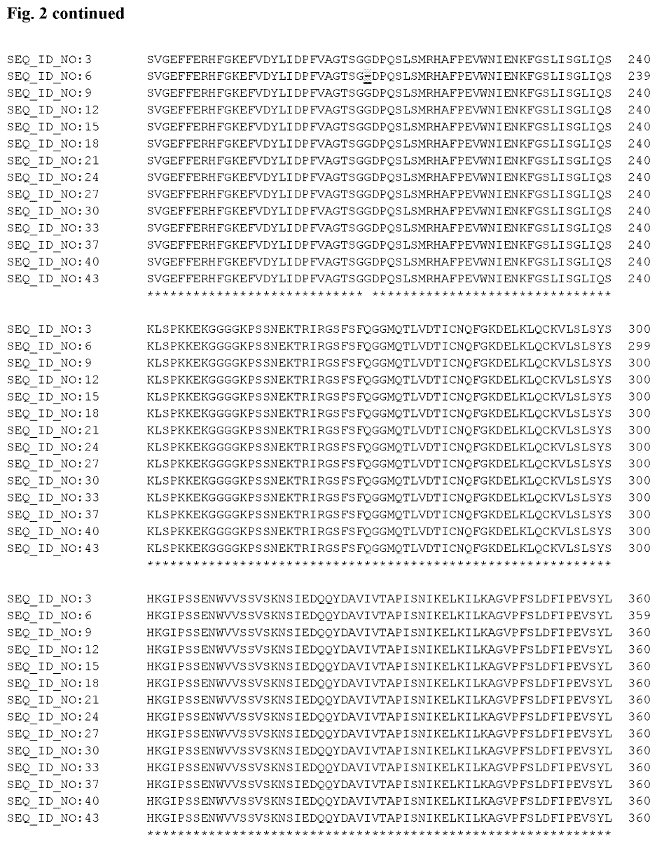

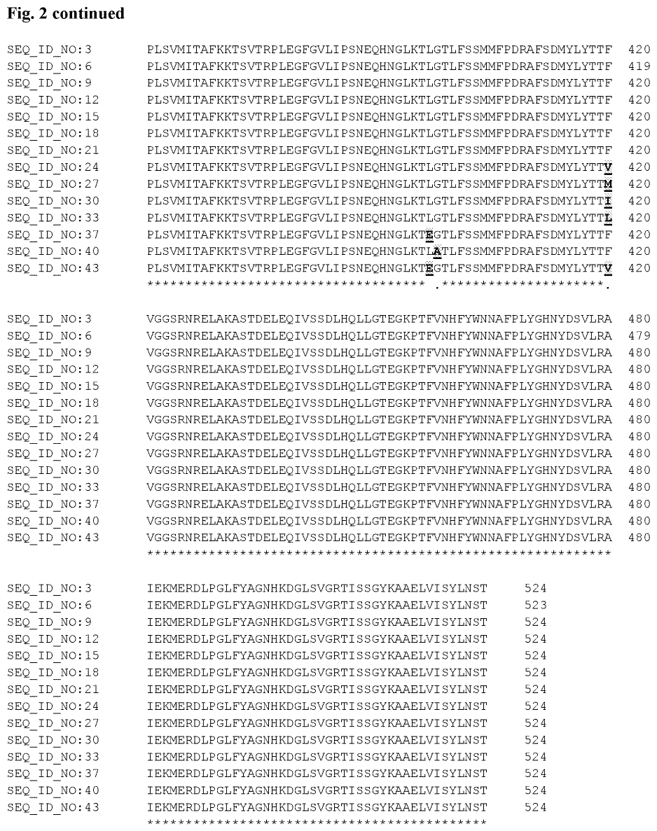

As shown in FIG. 1, a deletion of glycine at amino acid position no. 208 or 209 of the wild type Beta vulgaris PPO2 protein sequence (SEQ ID NO: 3) may confer resistance to a PPO herbicide. In some embodiments, the glycine at position no. 208 is deleted. In some aspects, the glycine at position no. 209 is deleted.

In some aspects, the deletion of glycine at position no. 208 or 209 is combined with one or more of the genetic alterations described below in Table 3.

TABLE 3
Additional genetic alterations

| | Amino acid |
|---|---|
| Genetic alterations to combine with G208/209 | position |
| deletion: | (FIG. 1) |
| Substitution of arginine for alanine, glycine, | 126 |
| leucine, isoleucine, or methionine | |
| Substitution of leucine for glutamic acid and | 397/420 |
| substitution at 420 as described below | |
| Substitution of phenylalanine for valine, | 420 |
| leucine, isoleucine, or methionine | |
| Substitution of glycine for alanine | 398 |

The disclosure provides novel, engineered proteins and the recombinant DNA molecules that encode them. As used herein, the term “engineered” refers to a non-natural DNA, protein, cell, or organism that would not normally be found in nature and was created by human intervention. An “engineered protein”, “engineered enzyme”, or “engineered PPO,” refers to a protein, enzyme, or PPO whose amino acid sequence was conceived of and created in the laboratory using one or more of the techniques of biotechnology, protein design, or protein engineering, such as molecular biology, protein biochemistry, bacterial transformation, plant transformation, site-directed mutagenesis, directed evolution using random mutagenesis, genome editing, gene editing, gene cloning, DNA ligation, DNA synthesis, protein synthesis, and DNA shuffling. For example, an engineered protein may have one or more deletions, insertions, or substitutions relative to the coding sequence of the wild-type protein and each deletion, insertion, or substitution takes place on one or more amino acids. Genetic engineering can be used to create a DNA molecule encoding an engineered protein, such as an engineered PPO that is herbicide tolerant and comprises at least one amino acid substitution or deletion relative to a wild-type PPO protein as described herein.

In some embodiments, the engineered proteins are genetically engineered with a targeted genome or gene editing system such as CRISPR-Cas system described below.

In some embodiments, provided is novel, engineered proteins that are herbicide-tolerant protoporphyrinogen oxidases (PPOs), as well as the recombinant, engineered DNA molecules encoding the herbicide-tolerant PPOs, compositions comprising the herbicide-tolerant PPO, and methods of using the herbicide-tolerant PPOs for weed control.

In some embodiments, engineered proteins (e.g. PPO2) provided by the disclosure have herbicide-tolerant protoporphyrinogen oxidase activity. As used herein, “herbicide-tolerant protoporphyrinogen oxidase” means the ability of a protoporphyrinogen oxidase to maintain at least some of its protoporphyrinogen oxidase activity in the presence of one or more PPO herbicide(s).

The term “protoporphyrinogen oxidase activity” means the ability to catalyze the six-electron oxidation (removal of electrons) of protoporphyrinogen IX to form protoporphyrin IX, that is, to catalyze the dehydrogenation of protoporphyrinogen to form protoporphyrin.

Enzymatic activity of a protoporphyrinogen oxidase can be measured by any means known in the art, for example, by an enzymatic assay in which the production of the product of protoporphyrinogen oxidase or the consumption of the substrate of protoporphyrinogen oxidase in the presence of one or more PPO herbicide(s) is measured via fluorescence, high performance liquid chromatography (HPLC), or mass spectrometry (MS).

In some embodiments, the disclosure provides recombinant constructs comprising recombinant polynucleotides encoding engineered herbicide-tolerant protoporphyrinogen oxidases for expression in plants, parts and cells. In other embodiments, the disclosure provides engineered proteins having herbicide-tolerant protoporphyrinogen oxidase activity. In further embodiments, the disclosure provides methods and compositions for using protein engineering and bioinformatics tools to obtain and improve herbicide-tolerant protoporphyrinogen oxidases. The disclosure further provides methods and compositions for producing plants, parts and cells tolerant to PPO herbicides, and methods of weed control using the cells, plants, and seeds.

Examples of engineered proteins provided herein are herbicide-tolerant PPOs comprising (i) one or more amino acid substitution(s) selected from R126A, R126G, R126L, R126I, R126M, L397E, G398A, F420V, F420M, F420I, and F420L, and (ii) one or more amino acid deletion(s) selected from G208 and G209, including all possible combinations thereof, wherein the position of the amino acid substitution(s) and/or deletion(s) are relative to the amino acid position set forth in SEQ ID NO: 3. In specific embodiments, an engineered protein provided herein comprises one, two, three, four, or more of any combination of such substitutions and/or deletions described herein.

Because of the degeneracy of the genetic code, a variety of different DNA sequences can encode the altered or engineered proteins disclosed herein. DNA sequences encoding PPO enzymes with the amino acid substitutions and deletions described herein can be produced by introducing mutations into the DNA sequence encoding a wild-type PPO enzyme using methods known in the art. It is well within the capability of one of skill in the art to create alternative DNA sequences encoding the same, or essentially the same, altered or engineered proteins as described herein. These variant or alternative DNA sequences are within the scope of the embodiments described herein. As used herein, references to “essentially the same” sequence refers to sequences which encode amino acid substitutions, deletions, additions, or insertions that do not materially alter the functional activity of the protein encoded by the DNA molecule of the embodiments described herein. Allelic variants of the nucleotide sequences encoding a wild-type or engineered protein are also encompassed within the scope of the embodiments described herein.

The above referenced genomic alterations may be achieved by any number of means well known in art, for example by genome modification using site-specific integration or genome editing. Targeted modification of plant genomes through the use of genome editing methods can be used to create improved plant lines through modification of plant genomic DNA. As used herein “site-directed integration” or “site-specific integration” refers to genome editing methods the enable targeted insertion of one or more nucleic acids of interest into a plant genome. Suitable methods for altering a wild-type DNA sequence or a preexisting transgenic sequence or for inserting DNA into a plant genome at a pre-determined chromosomal site include any method known in the art. Exemplary methods include the use of sequence specific nucleases, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system). Several embodiments relate to methods of genome editing by using single-stranded oligonucleotides to introduce precise base pair modifications in a plant genome, as described by Sauer et al., Plant Physiology 170(4): 1917-1928 (2016). Methods of genome editing to modify, delete, or insert nucleic acid sequences into genomic DNA are known in the art.

In some aspects, the present disclosure provides modification or replacement of an existing coding sequence, such as a PPO coding sequence or another existing transgenic insert, within a plant genome with a sequence encoding an engineered protein, such as an engineered PPO coding sequence of the present disclosure. Several embodiments relate to the use of a known genome editing methods, such as zinc-finger nucleases, engineered or native meganucleases, TALE-endonucleases, or an RNA-guided endonuclease (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system).

### Genome Editing by CRISPR

Conventional approaches to engineer herbicide resistance rely either on mutation breeding or introduction of novel genes into the genomes of crop species by transformation.

Conventional plant transformation methods deliver exogenous DNA that integrates into the genome at random locations. Thus, to identify and isolate transgenic lines with desirable traits, it is necessary to generate and screen thousands of random-integration events. Using genome editing, DNA can be modified in a targeted way providing new alternatives to develop novel traits in plants.

Genome editing by CRISPR, which stands for Clustered Regularly Interspaced Short Palindromic Repeats, is based on a natural immune process used by bacteria to defend themselves against invading viruses. Indeed, in bacteria the invading viral DNA will be cut through use of a guide RNA (gRNA), or piece of RNA, and a CRISPR-associated protein (Cas). The last step of the bacterial immune process, when the gRNA is combined with Cas and cleaves the target DNA, has been adopted for genome editing in laboratories.

There are at least three main CRISPR system types (Type I, II, and III) and at least 10 distinct subtypes (Makarova, K. S., et.al., Nat Rev Microbiol. 2011 May 9; 9(6):467-477). Type I and III systems use Cas protein complexes and short guide polynucleotide sequences to target selected DNA regions. Type II systems rely on a single protein (e.g. Cas9) and the targeting guide polynucleotide, where a portion of the 5′ end of a guide sequence is complementary to a target nucleic acid. For more information on the CRISPR gene editing compositions and methods of the present disclosure, see U.S. Pat. Nos. 8,697,359; 8,889,418; 8,771,945; and 8,871,445, each of which is hereby incorporated in its entirety for all purposes.

CRISPR genome editing requires two components, a gRNA and a Cas enzyme. These components associate to form a ribonucleoprotein (RNP) complex, where after the gRNA can base pair with a complementary protospacer sequence (i.e. the target genomic sequence of about 20 bases in length) under the condition that a particular adjacent sequence, called a protospacer-adjacent motif (PAM), is present in the genome. The PAM is only a few bases long, and its sequence depends on the type of Cas enzyme used. Once the gRNA binds to the target DNA (protospacer), the Cas enzyme recognizes this complex and makes a precise cut at the target site.

Either Cas9 or Cas12a (also called Cpf1) can be used to cleave target DNA, resulting in a Double Strand Break (DSB). Each Cas enzyme is directed by the gRNA to a user-specified cut site in the genome. Like Cas9 nucleases, Cas12a1 family members contain a RuvC-like endonuclease domain, but lack the second HNH endonuclease domain of Cas9. Cas12a cleaves DNA in a staggered pattern in contrast to Cas9 which produces a blunt-end. Moreover, for cleavage Cas12a requires only one RNA rather than the two tracrRNA and crRNA needed by Cas9. For Cas9 as well as Cas12a, the target sequence of the gRNAs must be next to a PAM sequence. In the case of Cas9, the PAM sequence corresponds to NGG, where N is any base.

The gRNA will recognize and bind to 20 nucleotides on the DNA strand opposite from the NGG PAM site. For Cas12a, the PAM sequence is TTTV, where V can represent A, C, or G.

Using Alt-R Cas12a Ultra from Integrated DNA Technologies, a TTTT PAM sequence may also work. The “V” of the TTTV is immediately adjacent to the base at the 5′ end of the non-targeted strand side of the protospacer element. The guide RNA for Cas12a is relatively short and is approximately 40 to 44 bases long.

The damage caused by the double strand break (DSB) will be repaired in eukaryotic cells, primarily by two pathways: Non-Homologous End-Joining (NHEJ) and Homology Directed Repair (HDR). The HDR mechanism requires the presence of a donor DNA template containing regions of homology to both sites of the DNA break. This donor DNA can carry specific mutations and has to be delivered simultaneously with a preassembled Cas RNP complex composed of Cas9 or Cas12a and synthetically produced gRNAs.

Altogether, targeted cleavage events induced by nucleases can be used to introduce targeted mutations (deletions, substitutions and insertions) in genomic DNA sequences and as such, can be used as an efficient tool for genome editing in plants including sugar beet and fodder beet.

In some aspects, the present disclosure relates to a recombinant DNA construct comprising an expression cassette(s) encoding a site-specific nuclease and, optionally, any associated protein(s) to carry out genome modification. These nuclease-expressing cassette(s) may be present in the same molecule or vector as a donor template for templated editing or an expression cassette comprising nucleic acid sequence encoding a PPO protein as described herein or on a separate molecule or vector. Several methods for site-directed integration are known in the art involving different sequence-specific nucleases (or complexes of proteins or guide RNA or both) that cut the genomic DNA to produce a double strand break (DSB) or nick at a desired genomic site or locus. As understood in the art, during the process of repairing the DSB or nick introduced by the nuclease enzyme, the donor template DNA, transgene, or expression cassette may become integrated into the genome at the site of the DSB or nick. The presence of the homology arm(s) in the DNA to be integrated may promote the adoption and targeting of the insertion sequence into the plant genome during the repair process through homologous recombination, although an insertion event may occur through non-homologous end joining (NHEJ).

In some aspects, the endonuclease is selected from a meganuclease, a zinc-finger nuclease (ZFN), a transcription activator-like effector nucleases (TALEN), an Argonaute (non limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas12a (also known as Cpf1), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, Cpf1, CasX, CasY, homologs thereof, or modified versions thereof.

In some aspects, the site-specific genome modification enzyme is a recombinase. Non-limiting examples of recombinases include a tyrosine recombinase attached to a DNA recognition motif provided herein is selected from the group consisting of a Cre recombinase, a Gin recombinase, a Flp recombinase, and a Tnpl recombinase. In some aspects, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif provided herein is selected from the group consisting of a PhiC3 1 integrase, an R4 integrase, and a TP-901 integrase. In another aspect, a DNA transposase attached to a DNA binding domain provided herein is selected from the group consisting of a TALE-piggyBac and TALE-Mutator.

In some aspects, plants comprising one or more of the genetic alterations described herein may be selfed or crossed to produce lines that are homozygous for one or more of the genetic alterations described herein. In some aspects, the genetic alterations described herein may be transferred or introgressed to other beet varieties through conventional breeding schemes.

In some embodiments, the disclosure provides a guide RNA suitable for use in the CRISPR-Cas based genome editing system taught herein, wherein said guide RNA comprises a nucleic acid sequence selected from SEQ ID NOs: 67-80.

In some embodiments, the disclosure provides a donor DNA suitable for use in the CRISPR-Cas based genome editing system taught herein, wherein said donor DNA comprises a nucleic acid sequence selected from SEQ ID NOs: 48-66 and 90-96.

In some embodiments, the guide RNA and donor template are provided in a ribonucleoprotein (RNP) complex. In some embodiments, the guide RNA and donor template are provided in a plasmid.

In some embodiments, a CRISPR-Cas genome editing system comprising; (a) a first expression construct comprising a target locus-specific guide RNA (gRNA) and a donor template, wherein said guide RNA comprises a nucleic acid sequence selected from SEQ ID NOs: 67-80, and wherein said donor template is selected from SEQ ID NOs: 48-66 and 90-96; and (b) a second expression construct comprising a polynucleotide encoding a CRISPR-associated protein nuclease. The CRISPR-Cas based genome editing system comprises at least one gRNA, a donor template, PAM sequence, and CRISPR-associated nuclease selected from the group consisting of Cas9, Cas12, Cas13, CasX, and CasY.

In some embodiments, RNP or the first expression construct comprises a target locus-specific guide RNA (gRNA) selected from the group consisting of SEQ ID NOs: 69-71, and 74, and a donor template selected from the group consisting of SEQ ID NOs: 52-56, 58-59, 66 and 93.

In some embodiments, RNP or the first expression construct comprises a target locus-specific guide RNA (gRNA) selected from the group consisting of SEQ ID NOs: 75, 76, and 79, and a donor template selected from the group consisting of SEQ ID NOs: 51, 60, and 63.

In some embodiments, RNP or the first expression construct comprises a target locus-specific guide RNA (gRNA) selected from the group consisting of SEQ ID NOs: 67, 68, and 73, and a donor template selected from the group consisting of SEQ ID NOs: 62, 64, and 92.

In some embodiments, RNP or the first expression construct comprises a target locus-specific guide RNA (gRNA) selected from the group consisting of SEQ ID NOs: 80 and 72, and donor template selected from the group consisting of SEQ ID NO: 61, 92, and 95.

In some embodiments, RNP or the first expression construct comprises a target locus-specific guide RNA (gRNA) selected from the group consisting of SEQ ID NOs: 77 and 78 and a donor template selected from the group consisting of SEQ ID NOs: 48-50, 57, 65, and 96.

In some embodiments, the genome editing method comprises the steps of: a) transfecting a protoplast obtained from Beta vulgaris cells with a genome editing system to generate a transfected protoplast, wherein the genome editing system comprises: i) a Cas enzyme;
ii) at least one guide RNA (gRNA), wherein the at least one gRNA targets a genomic region corresponding to between position 5457 and 5502 of SEQ ID NO: 1; and iii) at least one single-stranded donor DNA repair template designed to introduce a deletion of glycine at a position corresponding to 208 and/or 209 of SEQ ID NO: 3; b) exposing the transfected protoplast to a selective pressure of at least one herbicide that inhibits protoporphyrinogen oxidase; c) selecting a protoplast comprising a deletion of glycine at a position corresponding to 208 and/or 209 of SEQ ID NO: 3; and d) regenerating a plant from said selected protoplast to produce a Beta vulgaris plant with increased tolerance to an herbicide that inhibits protoporphyrinogen oxidase.

In some embodiments, the at least one gRNA targets a genomic region corresponding to between position 5483 and 5502, or between 5457 and 5479 of SEQ ID NO: 1.

Compositions and products for increased resistance to herbicide(s)

In some embodiments, the present disclosure provides a Beta vulgaris plant, or part thereof comprising a nucleic acid encoding a protoporphyrinogen oxidase 2 (PPO2) amino acid sequence. In some embodiments, said PPO2 amino acid sequence comprises a deletion of glycine at a position corresponding to 208 or 209 of SEQ ID NO: 3. In some embodiments, the deletion of glycine is at a position corresponding to 208 of SEQ ID NO: 3. In some embodiments, the deletion of glycine is at a position corresponding to 209 of SEQ ID NO: 3.

In some embodiments, the Beta vulgaris plant or part thereof further comprises a nucleic acid encoding a protoporphyrinogen oxidase 2 (PPO2) amino acid sequence comprising at least one of: (a) a substitution of arginine at a position corresponding to 126 of SEQ ID NO: 3; (b) a substitution of leucine at a position corresponding to 397 of SEQ ID NO: 3; (c) a substitution of glycine at a position corresponding to 398 of SEQ ID NO: 3; and (d) a substitution of phenylalanine at a position corresponding to 420 of SEQ ID NO: 3.

In some embodiments, the arginine at position 126 is replaced with alanine, glycine, leucine, isoleucine, or methionine.

In some embodiments, leucine at position 397 is replaced with glutamic acid.

In some embodiments, glycine at position 398 is replaced with alanine.

In some embodiments, phenylalanine at position 420 is replaced with valine, methionine, isoleucine, or leucine.

In some embodiments, the disclosure relates to a Beta vulgaris plant, or part thereof, comprising an engineered PPO2 protein having a deletion of glycine corresponding to position number 208 or 209 of SEQ ID NO: 3, and at least 90% identical to SEQ ID NO: 6. In some aspects, the sequence is 91%, 92%, 93%, 94%, 95%, 96%, 97%, 98%, or 99% identical to SEQ ID NO: 6.

In other embodiments, the disclosure relates to a Beta vulgaris plant, or part thereof, comprising a nucleic acid encoding a deletion of glycine corresponding to position number 208 or 209 of SEQ ID NO: 3, and a nucleic acid at least 90% identical to SEQ ID NO: 4, 97, or 104. In some aspects, the sequence is 91%, 92%, 93%, 94%, 95%, 96%, 97%, 98%, or 99% identical to SEQ ID NO: 4, 97, or 104.

In other embodiments, the disclosure relates to a Beta vulgaris plant, or part thereof, comprising a nucleic acid encoding a deletion of glycine corresponding to position number 208 or 209 of SEQ ID NO: 3, and a nucleic acid at least 90% identical to SEQ ID NO: 5, 98, or 105. In some aspects, the sequence is 91%, 92%, 93%, 94%, 95%, 96%, 97%, 98%, or 99% identical to SEQ ID NO: 5, 98, or 105.

In some embodiments, the plant or part thereof has a herbicide-tolerant protoporphyrinogen oxidase activity.

In some embodiments, the plant or part thereof has increased tolerance to a PPO herbicide when compared to a plant not having the amino acid deletion at a position corresponding to number 208 or 209 of SEQ ID NO: 3.

In some embodiments, the Beta vulgaris plant, or part thereof, is resistant or tolerant to a PPO herbicide selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

In some embodiments, the plant is a sugar beet or a fodder beet.

In some embodiments, the Beta vulgaris plant, or part thereof, further comprises a nucleic acid encoding a protein conferring resistance to a non-PPO herbicide, wherein the protein is 5-enolpyruvulshikimate-3-phosphate synthase (EPSPS) enzyme.

In some embodiments, the non-PPO herbicide is a glyphosate.

In some aspects, the glyphosate tolerance is conferred by the H7-1 event described in U.S. Pat. No. 7,335,816 and EP1597373 and obtainable from seed deposited with the NCIMB, Aberdeen (Scotland, U.K.) and having the accession number NCIMB 41158 or NCIMB 41159 or from various commercially available sugar beet varieties (see for example, oecd.org/agriculture/seeds/documents/codes-schemes-list-of-varieties-fodder-beet-and-sugar-beet.pdf, available on the world wide web).

In some embodiments, the nucleic acid is engineered with a targeted genome editing system, wherein the targeted gene editing system uses a clustered regularly interspaced short palindromic repeats (CRISPR)-Cas nuclease.

In some embodiments, the disclosure relates to an engineered polynucleotide comprising a nucleic acid sequence encoding a protein with a herbicide-tolerant protoporphyrinogen oxidase (PPO) activity. In some embodiments, the protein comprises at least one in-frame amino acid deletion at a position corresponding to 208 or 209 of SEQ ID NO: 3.

In some embodiments, the engineered polynucleotides encode proteins comprising one or more amino acid substitutions at positions corresponding to 126, 397, 398, and 420 of SEQ ID NO: 3.

In other embodiments, the disclosure relates to an engineered polypeptide having at least 90%, at least 91%, at least 92%, at least 93%, at least 94%, at least 95%, at least 96%, at least 97%, at least 98%, at least 99% sequence identity to SEQ ID NO: 6.

In further embodiments, the disclosure relates to an engineered polynucleotide comprising a nucleic acid sequence having at least 90%, at least 91%, at least 92%, at least 93%, at least 94%, at least 95%, at least 96%, at least 97%, at least 98%, at least 99% sequence identity to SEQ ID NO: 4, 97, or 104.

In further embodiments, the disclosure relates to an engineered polynucleotide comprising a nucleic acid sequence having at least 90%, at least 91%, at least 92%, at least 93%, at least 94%, at least 95%, at least 96%, at least 97%, at least 98%, at least 99% sequence identity to SEQ ID NO: 5, 98, or 105.

In some embodiments, the disclosure relates to DNA constructs comprising the engineered nucleotides disclosed herein. In some embodiments, the disclosure relates to methods of producing a plant having resistance to one or more PPO herbicides comprising transforming a plant or part thereof with a DNA construct comprising one or more of the guide or donor sequences disclosed herein.

Once a desired mutation is achieved, one skilled in the art can generate plants having various combinations of mutations in either a heterozygous, homozygous, or combination thereof, of the mutations disclosed herein through traditional breeding methods.

In some embodiments, the disclosure relates to a Beta vulgaris plant that is homozygous for one or more of the mutations disclosed herein. In some embodiments, the Beta vulgaris plant is heterozygous for one of the mutations disclosed herein. In some embodiments, the Beta vulgaris plant is heterozygous for two or more of the mutations disclosed herein. In some embodiments, the Beta vulgaris plant is homozygous for one or more mutations disclosed herein and also heterozygous for one or more additional mutations disclosed here.

### Methods for Controlling Undesired Plants

In some embodiments, the present disclosure teaches a method for controlling an undesired plant at a plant (e.g. Beta vulgaris) cultivation site. The method comprises providing a Beta vulgaris plant comprising a nucleic acid encoding an engineered PPO2 protein with herbicide-tolerant protoporphyrinogen oxidase (PPO) activity. In some embodiments, the protein comprises at least one in-frame amino acid deletion at a position corresponding to number 208 or 209 of SEQ ID NO: 3. The method further comprises applying to the site an effective amount of a PPO herbicide. In some embodiments, the PPO herbicide is selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

In some embodiments, the Beta vulgaris plant comprising a nucleic acid encoding an engineered PPO2 protein further comprises in cis or in trans at least one of (a) a substitution of arginine at a position corresponding to 126 of SEQ ID NO: 3; (b) a substitution of leucine at a position corresponding to 397 of SEQ ID NO: 3; (c) a substitution of glycine at a position corresponding to 398 of SEQ ID NO: 3; and (d) a substitution of phenylalanine at a position corresponding to 420 of SEQ ID NO: 3.

In some embodiments, the arginine at position 126 is replaced with alanine, glycine, leucine, isoleucine, or methionine.

In some embodiments, leucine at the position 397 is replaced with glutamic acid.

In some embodiments, glycine at the position 398 is replaced with alanine.

In some embodiments, phenylalanine at the position 420 is replaced with valine, methionine, isoleucine, and leucine.

In some embodiments, the disclosure relates to a method of producing a plant, plant part, or plant cell having resistance or tolerance to a PPO herbicide, the method comprising: transforming a plant, plant part, or plant cell with the recombinant, engineered polynucleotide taught herein. In some embodiments, the method comprises transforming the plant with one or more RNPs comprising a guide RNA and donor template described herein. In some embodiments, the method comprises transforming a plant with a DNA construct comprising a guide RNA and donor template described herein.

In some embodiments, the disclosure relates to a method for conferring PPO herbicide tolerance to a Beta vulgaris plant, part, or cell thereof comprising: expressing in said plant, part, or cell thereof the recombinant, engineered polynucleotide taught herein.

In some embodiments, the herbicide tolerance is to at least one PPO herbicide selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

In some embodiments, the plant, plant part, or plant cell is transformed with one or more additional desired traits. In some aspects, the one or more additional desired traits are introduced via a transgene. In some aspects, the one or more additional desired traits are introduced by direct or random mutagenesis. In some aspects, the one or more desired traits is introduced by introgression by one or more plant breeding techniques.

In some embodiments, the plant breeding techniques are selected from the group consisting of recurrent selection, mass selection, hybridization, open-pollination, backcrossing, pedigree breeding, mutation breeding, haploid/double haploid production, and marker enhanced selection. In some embodiments, the plant breeding technique is mutation breeding and the mutation selected is spontaneous or artificially induced.

In some embodiments, the one or more additional desired traits is resistance to a non-PPO herbicide. In some embodiments, the method of producing a plant tolerant to a PPO herbicide and at least one other herbicide comprises: a) obtaining a plant having an engineered PPO; b) crossing the plant with a second plant comprising tolerance to the at least one other herbicide, and c) selecting a progeny plant resulting from said crossing that comprises tolerance to a PPO herbicide and to at least one other herbicide.

### EXAMPLES

### Example 1: Generation of Sugar Beet Plants Having a G208/209 Deletion

For editing the sugar beet PPO2 gene, RNPs and donor templates were introduced into stomatal guard cell protoplasts isolated from well regenerating sugar beet genotype by means well known in the art (see for example, International Patent Publication WO/1995/010178). Components for RNPs and single strand HDR (ssHDR) donor templates were synthesized from Integrated DNA Technologies. In addition to introducing a point mutation causing a deletion of glycine at 209 of SEQ ID NO: 3, the donor template can further include modifications of the PAM site to prevent additional cuts. The RNPs were produced by assembling purified nuclease (S.p. Cas9 or A.s.Cas12a) and guide RNA (sgRNA or crRNA) for 10 minutes at 27 °C in a ratio 1/6. For generation of the G209 deletion mutation gRNA SEQ ID NO: 79 for Cas9 and SEQ ID NO:75 and 76 for Cpf1 enzymes were used.

Protoplasts were transfected with RNPs following a classical Polyethylene Glycol (PEG) transfection protocol (Hall, R. D., et al., 1996. A high efficiency technique for the generation of transgenic sugar beets from stomatal guard cells. Nature Biotechnology 14: 1133-1138). Protoplasts were then cultured on solid medium (polymer-containing medium, such as alginate or agarose-like containing medium), leading to the formation of microcalli.

To confirm the targeted mutagenesis can be achieved; a preliminary experiment was set up to estimate the percentage of targeted mutation. About 1 million protoplasts were transfected with selected guides and donor templates for each of the targeted mutations. After incubation for 1 day at 28 °C, DNA was extracted from the pool of 1 million guard cell protoplasts. Deep sequencing analysis was performed (Illumina NextSeq) on amplicons of 350 to 450 bp covering the targeted mutations. Reads were mapped to the publicly available sugar beet reference genome EL10_1.0 (McGrath et al, 2020. A contiguous de novo genome assembly of sugar beet EL10 (Beta vulgaris L.) bioRxiv), and variants were identified based on comparison to the reference sequence. For each of the amplicons, the frequency showing the expected edit (HDR), and the frequency showing another type of mutation (INDEL) is given in Table 4 below. Percentages were averaged over two replicates (independent transfections).

TABLE 4
Percentage of reads showing the expected mutation (HDR)
or another edit (INDEL) at position 126

| | | % HDR | % indels |
|---|---|---|---|
| | Amino Acid | PPLs | PPLs |
| | position of mutation | (n = 2) | (n = 2) |
| Cas9 | R126 | 0.16 | 9.6 |
| | G209del | 0.91 | 8.6 |
| | G398 | 0.16 | 12 |
| | L397-F420 | 0.02 | 21 |
| Cpf1 | R126 | 1.25 | 1.4 |
| | G208/209del | 1.6 | 2.2 |
| | G398 | 0.14 | 5.2 |
| | L397-F420 | 0.009 | 2.9 |

### Example 2: Screening for Resistance to PPO Herbicides

Screening for resistance to PPO herbicides can be done at several different time points. In some experiments, exposure of calli to different concentrations of PPO inhibitors such as saflufenacil (SAF) at a concentration that is lethal to more than 99% of the cells enables the selection of mutated cells being resistant to this herbicide molecule. For this, protoplasts are transferred on solid culture medium in the dark with addition after one week of culture of 10 μM Saflufenacil (SAF). SAF in dark conditions should not show any toxicity effect. Emerging calli are then selected and transferred to light conditions on medium containing 0.75 μM SAF, or SAF at a concentration at which wild-type calli are very sensitive. Thus, surviving calli would likely exhibit good resistance to PPO inhibitors. Additionally, the surviving calli may show somatic embryogenesis and will regenerate into sugar beet plantlets.

In other experiments, different SAF concentrations are used throughout the regeneration. In one part of the experiment above, protoplasts are transferred to solid culture medium with addition after one week of culture of 10-25 μM Saflufenacil (SAF). Emerging calli are selected and transferred to light conditions on medium with 0.75 μM SAF. After two weeks of growth, calli were transferred to regeneration medium containing 0.05 μM SAF for 4 weeks. SAF is then removed from the media. In another part of the experiments, SAF (0.2 μM or 0.5 μM) is only applied to the media for regenerating calli, this under light conditions approximately seven weeks after protoplast isolation.

In other experiments, friable calluses are isolated from explants created by the methods disclosed herein using a sterile scalpel and transferred to petri dishes containing solid PCM Simultaneously, the same can be done for friable calluses carrying the wild type allele of the PPO2 gene. After three weeks, the calluses are cut in half using a sterile scalpel and one half is transferred to a new petri dish containing solid PCM media supplemented with 500 mg/l claforan, while the other one is transferred to PCM media supplemented with 0.7872 mg/l lactofen and 500 mg/l claforan. The plates are scored for difference in phenotype weekly for 8 weeks, with the calluses being transferred to fresh plates every second week.

In other experiments, screening for resistance to PPO herbicides is done on regenerated sugar beet plants obtained after transfection of guard cell protoplasts by growing these plants on media containing SAF at a concentration of 2.5 μM. Visual observation of growth is done after 20 to 28 days. Plants are scored visually according to their growth, and marked “OK” for plants showing growth, “Bit growth” for plants showing limited growth, “No” for plants showing no growth, or “Dead” for plants that are dead after SAF exposure. The concentration of SAF used in this screen was identified as impacting severely on regenerated wild-type plants obtained after protoplast isolation and regeneration from the sugar beet genotype, from which guard cell protoplasts are isolated for transfection. Given the severe growth retardation and in some cases lethal effect of this SAF concentration on the wild-type genotype, edited plants scored as “OK” and “Bit growth”, show a good level of SAF tolerance and are selected for rooting.

In other experiments, plants carrying the mutations described above in Table 4 are acclimated in soil for 3-5 weeks and screened for PPO resistance with a spray test of the PPO herbicides Flumioxazin, Pyraflufen-ethyl, Saflufenacil, Bifenox, Trifludimoxazin and Carfentrazone-ethyl at a concentration of 0.5× to 1× of the recommended dose. The phytotoxicity effect of PPO inhibiting herbicides can be measured in mutated sugar beet plants based on the scale from 0% to 100% (i.e. 0% being no damage/no phytotoxicity observed and 100% being plants were completely killed) compared to wild-type plants.

In order to establish a baseline of the native PPO herbicide tolerance among the non-edited sugar beet lines, LD50 (lowest dose where no more than 50% seedlings show moderate levels of herbicide toxicity) and LD100 (lowest dose that is lethal for all tested seedlings) is determined in the following way: individuals from each inbred line are germinated in soil in the greenhouse under artificial light (20 hours) at 20/15 °C temperature day/night respectively.

Fourteen days after seedling emergence, the seedlings are sprayed with a specified dose of active compounds from mixtures containing PPO targeting herbicide compounds. The seedlings are scored for survival one, two and three weeks after spraying. The same setup is repeated for all sugar beet lines, doses and mixtures.

Edited lines of sugar beet at the TO stage (first plants after nucleotide changes have been introduced) carrying the edited gene in homozygous or heterozygous hemizygous state are demonstrated to be tolerant against the PPO targeting herbicide if they do not show any signs of growth retardation, loss of chlorophyll or other visual signs of damage.

At the T1 stage (one generation after seed production), the edited lines are phenotypically evaluated for agronomically valuable traits. This is done by sowing plants from each individual event in soil in the greenhouse under artificial light (20 hours) at 20/15 °C temperature day/night respectively, and continuously monitoring their development. The traits analyzed include, but are not limited to, germination frequency, germination rate, seedling development, photosynthetic activity, root development, shoot growth rate, sugar content, root yield and time to flowering. The corresponding non-edited lines are used as controls.

### Example 3: Generation of Sugar Beet Plants Having a G208 Deletion

For editing the sugar beet PPO2 gene, RNPs and donor templates were introduced into stomatal guard cell protoplasts isolated from well regenerating sugar beet genotype by means well known in the art (see for example, International Patent Publication WO/1995/010178).

Components for RNPs and single strand HDR (ssHDR) donor template (SEQ ID NO: 90-SEQ ID NO: 94) were synthesized from Integrated DNA Technologies. In addition to introducing the deletion at G208 of SEQ ID NO: 3, the donor template can further include modifications of the PAM site and/or in the seed region of the guide RNA (from CAGTCT to CAATCC, SEQ ID NO: 90, from CACAAAAGG to CACGAAGGG, SEQ ID NO: 94) to prevent additional cuts. The RNPs were produced by assembling purified nuclease (S.p. Cas9 or L.b. Cas12a) and guide RNA (sgRNA or crRNA) for 10 minutes at 27 °C in a ratio 1/6. For generation of the G208 deletion gRNA SEQ ID NO:79 for Cas9 and SEQ ID NO:76 for Cpf1 enzymes were used.

Protoplasts were transfected with RNPs following a classical Polyethylene Glycol (PEG) transfection protocol (Hall, R. D., et al., 1996. A high efficiency technique for the generation of transgenic sugar beets from stomatal guard cells. Nature Biotechnology 14: 1133-1138). Protoplasts were then cultured on solid medium (polymer-containing medium, such as alginate or agarose-like containing medium), leading to the formation of microcalli. Screening for resistance to PPO herbicides has been done at several different time points.

In some experiments, exposure of calli to different concentrations of PPO inhibitors such as saflufenacil (SAF) at a concentration that is lethal to more than 99% of the cells enabled the selection of mutated cells being resistant to this herbicide molecule. For this, protoplasts were transferred on solid culture medium in the dark with addition after one week of culture of 10 μM Saflufenacil (SAF). SAF in dark conditions should not show any toxicity effect.

Emerging calli were then selected and transferred to light conditions on medium containing 0.75 μM SAF, or SAF at a concentration at which wild-type calli are very sensitive. Thus, surviving calli would likely exhibit good resistance to PPO inhibitors. Additionally, the surviving calli may show somatic embryogenesis and will regenerate into sugar beet plantlets.

After two weeks of growth, calli were transferred to regeneration medium containing 0.05 μM SAF for 4 weeks. SAF is then removed from the media.

Screening for resistance to PPO herbicides was done on regenerated sugar beet plants obtained after transfection of guard cell protoplasts by growing these plants (8 clones or copies per plant) on media containing SAF at a concentration of 2.5 μM. Visual observation of growth was done after 20 to 28 days. Plants were scored visually according to their growth, and marked “OK” for plants showing growth, “Bit growth” for plants showing limited growth, “No” for plants showing no growth, or “Dead” for plants that were dead after SAF exposure.

The concentration of SAF used in this screen was identified as impacting severely on regenerated wild-type plants obtained after protoplast isolation and regeneration from the sugar beet genotype, from which guard cell protoplasts are isolated for transfection.

Given the severe growth retardation and in some cases lethal effect of this SAF concentration on the wild-type genotype, edited plants scored as “OK” showed a good level of SAF tolerance and were selected for rooting. All plant events shown below in Table 5 were heterozygous for the G208 deletion.

TABLE 5
Growth of plants carrying the G208 deletion on SAF

| | | | Growth |
|---|---|---|---|
| | | | SAF |
| Events | HDR/INDEL | Enzyme/mutation | 2.5 μM |
| HTD164 | HDR | Cas9/G208del | OK |
| HTH153 | HDR | Cpf1/G208del | OK |
| HTH191 | HDR | Cpf1/G208del | OK |
| HTH192 | HDR | Cpf1/G208del | OK |
| HTH195 | HDR | Cpf1/G208del | OK |
| HTH197 | HDR | Cpf1/G208del | OK |
| HTH240 | HDR | Cpf1/G208del | OK |
| HTH244 | HDR | Cpf1/G208del | OK |
| HTH247 | HDR | Cpf1/G208del | OK |
| HTH251 | HDR | Cpf1/G208del | OK |
| HTH256 | HDR | Cpf1/G208del | OK |

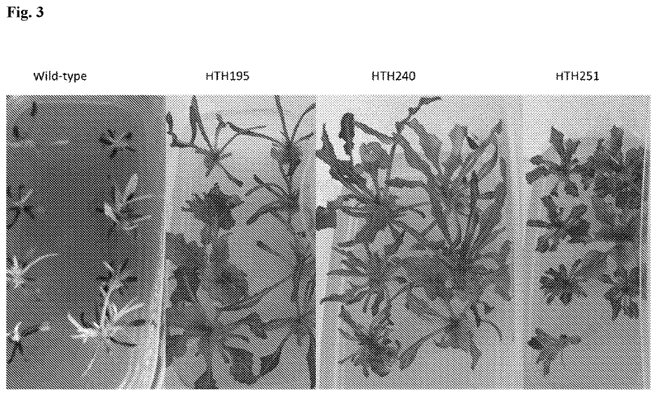

FIG. 3 shows copies of three different edited sugar beet genotypes, HTH195, HTH240 and HTH251 with the expected mutation (G208del) on SAF 2.5 μM containing media after 20 days. Clear differences in growth and plant development can be observed between the edited and wild-type plants on this media.

### Example 4: Screening Sugar Beet Plantlets Carrying the G208 Deletion

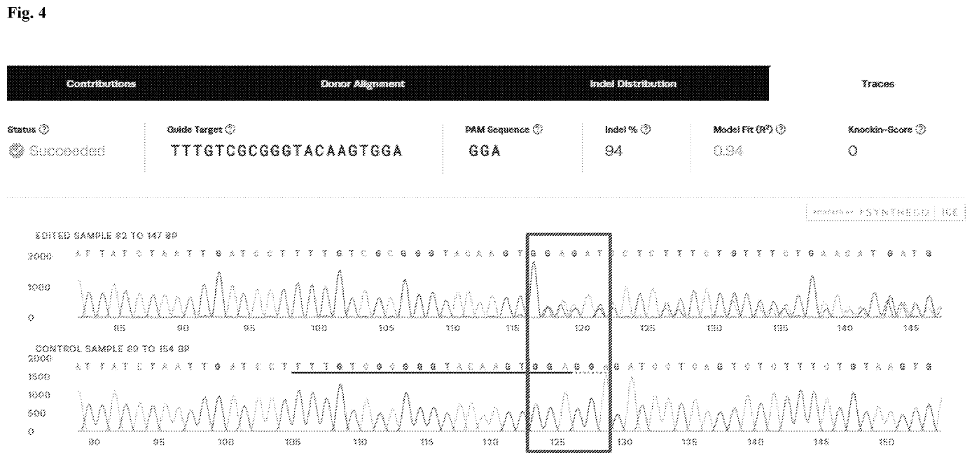

To check for the presence of the expected mutation in regenerated plants KASP assays were developed and used for screening. Leaf samples were collected from the regenerated plants and analyzed for the presence of the expected mutation using KASP analysis (LGC Genomics). KASP assays S01, S02 and S03 were used to detect the mutation without silent mutations in the template, while S04 and S05 were used to detect the mutation with silent mutations in the template. In all assays primer X will amplify the mutated allele, while Y will amplify the wild-type allele. S01, S02, and S04 have been designed in the reverse orientation, while S03 and S05 have been designed in the forward orientation. S01 differs from the others in the location of the common primer which is more closely located to the mutated site. Plants showing the expected deletion indicated by KASP were then confirmed using Sanger sequencing analysis using SEQ ID NOs: 121 and 122 (FIG. 4). Chromatograms were visually analyzed to identify the expected mutation; an estimation of the frequency of the edits was performed using the ICE (Inference of CRISPR Edits) algorithm (Conant et al, 2022. Inference of CRISPR Edits from Sanger Trace Data. The CRISPR Journal 2: 123-130). In all 45 edited plants showing the expected deletion at 208 mutation, it was combined with either a wild-type sequence, or a differently edited allele.

### Example 5: In Planta Screening for Resistance to PPO Herbicides

In other experiments, plants carrying the mutation G208/209 deletion described below in Table 6 were acclimatized in soil for 3-5 weeks and screened for PPO resistance with a spray test of the PPO herbicides Saflufenacil (Brand name TREEVIX at a recommended dose of 25 g a.i./ha), and Pyraflufen-ethyl (Brand name EVOLUTION at a recommended dose of 0.81/ha, or a.i. 26.5 g/l). The spray was performed at a concentration of 2× and 1× of the recommended dose for Saflufenacil), and 0.2× for Pyraflufen-ethyl). Water treatment was included as a control.

Eight different sugar beet plants (HTD159, HTD164, HTH153, HTH191, HTH192, HTH195, HTH197, HTH214) carrying edits at the position G208/209, from which 7 showed the expected deletion of glycine at position 208/209 (HTD164, HTH153, HTH191, HTH192, HTH195, HTH197, HTH214) were used in one experiment. In another experiment 6 different sugar beet plants (HTH246, HTH247, HTH250, HTH256, HTH257, HTH259) carrying the edits at position 208/209, from which 5 showed the expected deletion of glycine at position 208/209 (HTH246, HTH247, HTH256, HTH257, HTH259) were used. Sugar beet HTH250 comprised a deletion of TGG (the thymine just prior to the two guanines in the targeted GGA) instead of GGA.

In both experiments each sugar beet plant was multiplied in vitro as to include a total of minimum 4 clones or replicates of each edited sugar beet plant per herbicide treatment.

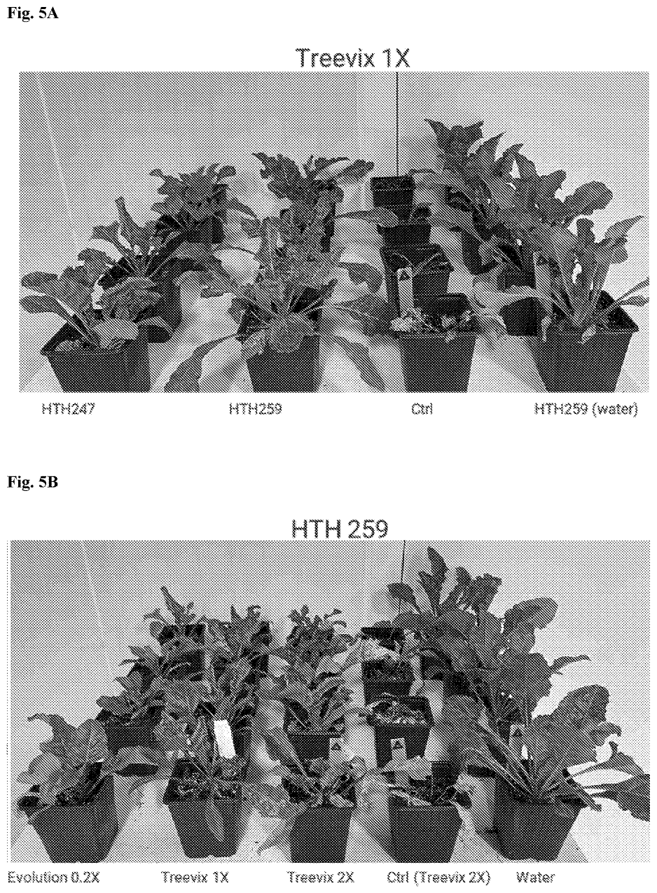

A subset of these treated plants is shown in FIGS. 5A and 5B. In FIG. 5A, differences in response between edited plants and the non-edited elite sugar beet genotype (Ctrl) can be observed 21 days after spray with Treevix 1×. FIG. 5B shows the phytotoxicity effect of Treevix 1×, 2× on an elite sugar beet genotype, compared to the edited sugar beet plant HTH259 showing the expected edit.

Table 6 shows the phytotoxicity effect of PPO inhibiting herbicides measured in mutated sugar beet plants (on a set of minimum 4 clones per edited sugar beet plant) based on a scale from 0% to 100%, or 1 to 5, (i.e. 0% or 1 being no damage/no phytotoxicity observed and 100% or 5, being all plants were completely damaged or killed,) compared to wild-type plants, 21 days after spraying with pyraflufen-ethyl herbicide (Evolution 0.2× of the recommended dose corresponding to 0.8 l/ha, or a.i. 26.5 g/l), and saflufenacil herbicide (Treevix 1× and 2×, 25 g a.i./ha and 50 g a.i./ha respectively). No score is provided when the plant has not been tested, noted as NT in Table 6. Scores were given on group of 4 copies per edited plant. HDR=edited plant showing the expected mutation, INDEL=edited plant showing another type of edit than the expected mutation. Plants were scored 3, 7, 14, 17, and 21 Days after spray.

TABLE 6
Resistance to PPO targeting herbicides

| | | Treevix | Treevix | Evolution | |
|---|---|---|---|---|---|
| Event | HDR/INDEL | 2X | 1X | 0, 2X | Water |
| HTD159 | INDEL | NT | NT | NT | NT |
| HTD164 | HDR | 4 | 3 | 3 | 1 |
| HTH153 | HDR | 4 | 3 | 2 | 1 |
| HTH191 | HDR | 3 | 2 | 2 | 1 |
| HTH192 | HDR | 3 | 1 | 1 | 1 |
| HTH195 | HDR | 4 | 2 | 1 | 1 |
| HTH197 | HDR | 3 | 2 | 1 | 1 |
| HTH214 | HDR | 3 | 2 | 1 | 1 |
| HTH246 | HDR | 2 | 2 | 2 | 1 |
| HTH247 | HDR | 3 | 2 | 2 | 1 |
| HTH250 | INDEL | 4 | 4 | 5 | 1 |
| HTH256 | HDR | 3 | 2 | 3 | 1 |
| HTH257 | HDR | 2 | 2 | 2 | 1 |
| HTH259 | HDR | 3 | 2 | 2 | 1 |
| Ctrl | | 5 | 5 | 5 | 1 |

### Example 6: Genetically Engineering the Endogenous Sugar Beet PPO2 Gene

Sequence analysis of PPO2 of four different sugar beet genotypes showed the absence of any polymorphism on the protein level (two public ref genomes, two proprietary ref genomes). Potential mutations that may convey resistance to PPO herbicides were identified and are shown below in Table 7.

Plants will first be generated carrying in-frame deletions of glycine at position 208 or 209. Plants may further comprise additional edits as described in Table 7 below at positions 126, 397/420, 398, and 420 in the PPO2 gene (Butterfass chromosome 9) of sugar beet (Beta vulgaris L.).

TABLE 7
Example amino acids subsitutions in the sugar beet PPO2 sequence

| Position | | | | |
|---|---|---|---|---|
| in SEQ | Example | Type of | Possible nucleotide | Example Donor |
| ID NO: 3 | sgRNA | mutation | change | DNA |
| 208 | SEQ ID NO: 75 | Gly deletion | Deletion of GGA | SEQ ID NO: 51 |
| | SEQ ID NO: 76 | | | SEQ ID NO: 60 |
| | SEQ ID NO: 79 | | | SEQ ID NO: 63 |
| 209 | SEQ ID NO: 75 | Gly deletion | Deletion of GGA | SEQ ID NO: 51 |
| | SEQ ID NO: 76 | | | SEQ ID NO: 60 |
| | SEQ ID NO: 79 | | | SEQ ID NO: 63 |
| | | | | SEQ ID NO: 90 |
| | | | | SEQ ID NO: 94 |
| 126 | SEQ ID NO: 69 | Arg-Ala | AGA to GCT, | SEQ ID NO: 52 |
| | SEQ ID NO: 70 | | GCC, GCA, or | |
| | SEQ ID NO: 71 | | GCG | |
| | SEQ ID NO: 74 | Arg-Gly | AGA to GGT, | SEQ ID NO: 53 |
| | | | GGC, GGA, or | |
| | | | GGG | |
| | | Arg-Leu | AGA to TTA, TTG, | SEQ ID NO: 54 |
| | | | CTT, CTC, CTA, or | SEQ ID NO: 58 |
| | | | CTG | SEQ ID NO: 59 |
| | | | | SEQ ID NO: 66 |
| | | | | SEQ ID NO: 93 |
| | | Arg-Ile | AGA to ATT, ATC, | SEQ ID NO: 55 |
| | | | or ATA | |
| | | Arg-Met | AGA to ATG | SEQ ID NO: 56 |
| 397/420 | SEQ ID NO: 67 | Leu-Glu at | CTC to GAG or | SEQ ID NO: 62 |
| | SEQ ID NO: 68 | 397 | GAA | SEQ ID NO: 64 |
| | SEQ ID NO: 73 | Phe-Val at | TTT to GTT, GTC, | SEQ ID NO: 92 |
| | | 420 | GTA, or GTG | |
| 398 | SEQ ID NO: 72 | Gly-Ala | GGT to GCT, GCC, | SEQ ID NO: 61 |
| | SEQ ID NO: 80 | | GCA, or GCG | SEQ ID NO: 92 |
| | | | | SEQ ID NO: 95 |
| 420 | SEQ ID NO: 77 | Phe-Val | TTT to GTT, GTC, | SEQ ID NO: 57 |
| | SEQ ID NO: 78 | | GTA, or GTG | SEQ ID NO: 96 |
| | | Phe-Met | TTT to ATG | SEQ ID NO: 48 |
| | | Phe-Ile | TTT to ATT, ATC, | SEQ ID NO: 49 |
| | | | or ATA | |
| | | Phe-Leu | TTT to TTA, TTG, | SEQ ID NO: 50 |
| | | | CTT, CTC, CTA, or | SEQ ID NO: 65 |
| | | | CTG | |

Mutations at the positions shown above may be obtained individually or introduced simultaneously. Genomic edits may be achieved by any means known in the art, for example, by CRISPR. Delivery of CRISPR-Cas RNPs (Ribonucleoprotein comprised of sgRNA+DNA endonuclease) to sugar beet protoplasts targeting the gene encoding protoporphyrinogen oxidase (PPO2) can be directly implemented using previously developed protoplast isolation, transfection and regeneration protocols. Example sgRNAs are provided in SEQ TD NO: 75, SEQ TD NO: 76, and SEQ TD NO: 79. Example donor DNA sequences for the amino acid deletion at position 208 or 209 are provided in SEQ TD NO: 51, SEQ TD NO: 60, SEQ TD NO: 63, SEQ TD NO: 90, and SEQ TD NO: 94 (see also Table 7 above).

Additional sgRNAs and donor DNAs for a substitution of the 126^{th} amino acid, a substitution of the 397^{th} and 420^{th} amino acids, a substitution of the 398^{th} amino acid, or a substitution of the 420^{th} amino acid of sugar beet PPO2 are also shown above in Table 7. After induced DNA cleavage these mutations are integrated through HDR in the PPO2 gene in sugar beet.

For example, one may use HDR, using Cas9 or Cas12a (Cpf1) enzymes and their respective donor DNA templates, for all the above-mentioned positions in Table 7. For the simultaneous introduction of the mutations (for example at positions 397 and 420), one would ideally use two guide RNA sequences in combination with one DNA repair template. However, for Cas12a guide RNA design was not possible at position 397 because of the absence of a PAM site close by, which might reduce the chances of achieving the combined mutation at 397 and 420 (397-420) when using Cas12a. Only one guide RNA was consequently designed to be used at position 420. For Cas9, guide RNAs could be designed at both 397 and 420, which increases the success to obtain the combined mutation through HDR.

### Example 7: Transformation with Plasmids and Regeneration of Sugar Beet from Leaf Tissue

Seeds from sugar beet cultivars will be sterilized by submersion in 70% hypochlorite solution for 15 minutes before being rinsed in 3 volumes of sterile water. After removing the water, the seeds will be submerged in 70% ethanol for 12 hours before being spread out on filter paper to dry completely. When the seeds are completely dry, they will be sown in sterile plant tissue jars containing half-strength Murashige and Skoog (MS) (Murashige and Skoog, 1962 “A Revised Medium for Rapid Growth and BioAssays with Tobacco Tissue Cultures”. Physiologia Plantarum 15 (3): sid. 473-497) media with 3% sucrose and allowed to germinate in an in-vitro chamber supplemented with artificial lights (200 μmol/m²/s, 18 hours, 22 °C). For transformation, Agrobacterium tumefaciens carrying the relevant constructs will be grown in liquid LB supplemented with relevant antibiotics until the cultures are in the exponential growth phase at which acetosyringone (Sigma-Aldrich, Saint Louis, MO, U.S.) to a final concentration of 100 μM will be added. One hour later, the cells will be harvested using gentle centrifugation and resuspended in co-cultivation buffer (5 mM 4-Morpholineethanesulfonic acid (Sigma-Aldrich, Saint Louis, MO, U.S.), 5 mM MgSO4 (Sigma-Aldrich, Saint Louis, MO, U.S.), pH 5.7, 100 μM acetosyringone (Sigma-Aldrich, Saint Louis, MO, U.S.)) to a final OD₆₀₀ of 0.2 to form the co-cultivation media.

After this, 1-2 mm wide and 5-15 mm long pieces of leaf from the sugar beet cultivars will be cut and immediately placed in petri dishes containing the co-cultivation media with abaxial side up. The leaf surface can be gently scarred using a sterile scalpel. After 1 minutes, the leaf tissue can be transferred to a solid co-cultivation media ( 1/10 MS, 3% sucrose ((Sigma-Aldrich, Saint Louis, MO, U.S.), pH 5.7) and cultivated in darkness for 3 days at 22 °C before being transferred to light conditions (200 μmol/m²/s, 18 hours). After two weeks, the transformed leaf tissue will be transferred to selection media (½ MS, 2% sucrose, 500 mg/l claforan (Sigma-Aldrich, Saint Louis, MO, U.S.), 0.25 mg/l 6-Benzylaminopurine (Sigma-Aldrich, Saint Louis, MO, U.S.), 0.05 mg/l 1-Naphthaleneacetic acid (Sigma-Aldrich, Saint Louis, MO, U.S.), 0.7872 mg/l lactofen (Sigma-Aldrich, Saint Louis, MO, U.S.) and 100 mg/l kanamycin (Sigma-Aldrich, Saint Louis, MO, U.S.)).

Generated shoots will be continuously removed and transferred to rooting media (½ MS, 2% sucrose, 10 mg/l Indole-3-butyric acid (Sigma-Aldrich, Saint Louis, MO, U.S.), 0.7872 mg/l lactofen and 500 mg/l claforan) until proper rooting has taken place, upon which the plants will be transferred to soil.

### Example 8: Transformation with Plasmids and Regeneration of Sugar Beet from Leaf Base Protoplasts

Seeds from cultivars of sugar beets can be sterilized and grown as described in Example 5 above. Approximately three weeks after germination, leaf material may be harvested by removing the top ⅔ portion of the leaf as well as the middle stem before cutting the remaining tissue into 1 mm wide and 15 mm long pieces using a sterile scalpel. The tissue is then immediately placed in a sterile 15 ml centrifuge tube with 5 ml PCWS and incubated in the dark at room temperature. After one hour, the PCWS is removed using a sterile pasteur pipette and the weight of the tissue determined. The tissue will then be transferred to a sterile petri dish and 5 ml of DCWS added for each gram of tissue, gently stirred and incubated in darkness at 22 °C for 18 hours. After incubation, the material is again gently stirred before being filtrated into a sterile 15 ml centrifuge tube through a 100 μm mesh to remove larger debris and centrifuged at 100×g for 10 minutes. The supernatant will be decanted and the remaining pellet resuspended in 15 ml PCWS. The protoplast suspension is then centrifuged 100×g for 10 minutes and the resulting band of protoplasts are extracted using a sterile pasteur pipette and resuspended in an equal volume of WCWS and centrifuged at 100×g for 10 minutes to wash the protoplasts. This washing can be repeated three times.

For PEG-mediated transformation of the isolated protoplasts, the protoplasts are washed using 0.45 M mannitol solution, and resuspended in one ml of transfection solution (015 mM MgCl₂·2H₂O, 0.45 M Mannitol, 10 mM 4-Morpholineethanesulfonic acid, pH 5.7). Protoplast concentration can be determined using a hematocytometer and the suspension diluted to a final concentration of 4×10⁵ cells/ml. For the transfection, 10 μg plasmid/4×10⁵ cells are carefully added to the protoplast suspension and the solution is gently mixed for three minutes before adding an equal volume of 40% PEG-solution very carefully. After 15 minutes, 3 ml of 0.45 M mannitol solution is added dropwise, the suspension centrifuged at 100×g for 10 minutes to collect the protoplasts and the supernatant decanted. The protoplasts are then embedded in alginate using a modified protocol from Damm et al. (1989) (“Regeneration of fertile plants from protoplasts of different Arabidopsis thaliana genotypes” Molecular and General Genetics 213: 15-20) in which the protoplasts first are suspended in alginate by mixing the protoplasts suspension with an equal volume of sodium alginate solution (2.8% sodium alginate (Sigma-Aldrich, Saint Louis, MO, U.S.) and 0.4 M mannitol). The suspension is then spread unto a solid agar medium containing 40 mM of CaCl₂. After one hour at room temperature the solidified discs containing the protoplasts will be transferred to a protoplast regeneration media (PRGM). After several weeks, friable microcalluses will be transferred to shoot inducing media.

### Example 9: Non-Transgenic Gene Editing with Cpf1

To achieve the above described edits in the sugar beet PPO gene, a protoplast based system can be utilized in such a way that the resulting edited individuals do not contain any transgenic regions.

First, sgRNA sequences comprising SEQ ID NOs: 70, 74, 75, 76, 77 and 78 can be cloned into a plant Cpf1-sgRNA expression vector downstream a CaMV 35S promoter. (1) Two sgRNAs (SEQ ID NOs: 70 and 74) are used for substitutions of Arg (R) to Ala (A), Gly (G), Leu (L), Ile (I), or Met (M) at position 126. (2) Two sgRNAs (SEQ ID NOs: 75 and 76) are used for deletion of Gly (G) at position 208 or 209. (3) Two sgRNAs (SEQ ID NOs: 77 and 78) are used for substitutions of Phe (F) to Val (V), Met (M), Ile (I) or Leu (L) at position 420.

The vector may also contain a codon-optimized Cpf1, SEQ ID NO: 34, under regulatory control of a CaMV 35S promoter. In parallel, the donor sequence comprising of SEQ ID NOs: 51 (for deletion of G at 209), 52-56 and 58 (for substitutions of R to A, G, L, I and M at 126), and 57, 48-50 (for substitutions of F to V, M, I and L at 420) are individually cloned into a donor vector. The Cpf1-sgRNA vector and one of the donor vectors are then transformed into sugar beet protoplasts isolated from each cultivar in the same way as described above using 10 μg of each plasmid. An aliquot of the transformed protoplasts is taken immediately before fixation in alginate and analyzed for efficiency using NGS. >1% of correct sequences is required for continued work. Once shoots have been formed, a tissue sample is taken and analyzed using Sanger sequencing for targeted mutations and for PCR-evaluation of transgene insertions. Only plants showing positive results for the targeted mutation and no transgene insert are transferred to the rooting step described above.

### Example 10: Gene Editing with Cpf1 Using a Transient Expression System

Another way of achieving the above described edits in the sugar beet PPO gene in such a way that the resulting edited individuals do not contain any transgenic regions is by using Agrobacterium-mediated transient expression. A vector as described above in Example 7 is generated containing an additional bacterial resistance marker gene cassette (kanamycin). In parallel, the donor sequence comprising of SEQ ID NOs: 51 (for deletion of G at 208/209), 52-56 and 58 (for substitutions of R to A, G, L, I and M at 126), and 57, 48-50 (for substitutions of F to V, M, I and L at 420 are individually cloned into a donor vectors containing a bacterial resistance marker gene cassette (spectinomycin).

These binary plasmids coding for planta expression of Cpf1 and the relevant guide-RNA/donor region are co-transformed into Agrobacterium rhizogenes K599 using electroporation. The A. rhizogenes are then grown in liquid LB supplemented with relevant antibiotics until the cultures are in the exponential growth phase at which acetosyringone to a final concentration of 100 μM is added. One hour later, the cells are harvested using centrifugation, resuspended in infection buffer (5 mM 4-Morpholineethanesulfonic acid, 5 mM MgSO4, pH 5.7, 100 μM acetosyringone) for a final OD₆₀₀ of 0.2 and then infilitrated into the lower part of the hypocotyl in young sugar beet seedlings. Hairy roots forming from the infected sites are then collected and placed onto solid media containing ½ MS supplemented with 200 mg/l claforan and 50 mg/l kanamycin. After two weeks, surviving tissue is screened for presence of target mutation and transgene inserts using sanger sequencing and fragment length PCR. Once positive tissue has been determined, it is transferred to shoot inducing media followed by root inducing media, using 0.7872 mg/l lactofen as selective agent for PPO herbicide resistance.

### Example 11: Gene Editing with Biolistic Transformation

Another way of achieving the targeted edits shown in Table 7 is through biolistic transformation of sugar beet calli. In this method, two different plasmids carrying and expressing in planta the Cpf1 and gRNA/complementary region respectively. Mixed in a 1:1 ratio, particle bombardment can be performed using a particle bombardment system (e.g. a Bio-Rad PDS1000/He at a target distance of 60 mm and at helium pressure 1100 psi) to introduce the plasmids into 1 month old calli. After 48 h aliquots can be taken to verify the efficiency of the method using PCR or NGS-based methods. Once efficiency has been determined, protoplasts are transferred to solid cultivation media that may or may not contain a PPO targeting herbicide. Regenerated plants are screened for the relevant edit using PCR or NGS-based approaches.

Table 8 below lists the expected genomic, cDNA, and protein sequences of lines generated carrying various mutations disclosed herein.

TABLE 8
Summary of Sequence Information

| | Sequence | | | |
|---|---|---|---|---|
| SEQ ID NO. | Type | Origin | Brief Description | Length |
| SEQ_ID_NO: 1 | DNA | Beta | Genomic DNA sequence of wild type Beta | 37009 |
| | | vulgaris | vulgaris PPO2 | |
| SEQ_ID_NO: 2 | cDNA | Beta | cDNA sequence of wild type Beta vulgaris PPO2 | 1575 |
| | | vulgaris | | |
| SEQ_ID_NO: 3 | protein | Beta | Protein sequence of wild type Beta vulgaris | 524 |
| | | vulgaris | PPO2 | |
| SEQ_ID_NO: 4 | DNA | Beta | Genomic DNA sequence of dG209 or dG208 | 36493 |
| | | vulgaris | mutant of Beta vulgaris PPO2 | |
| SEQ_ID_NO: 5 | cDNA | Beta | cDNA sequence of dG209 or dG208 mutant of | 1572 |
| | | vulgaris | Beta vulgaris PPO2 | |
| SEQ_ID_NO: 6 | protein | Beta | Protein sequence of dG209 or dG208 mutant of | 523 |
| | | vulgaris | Beta vulgaris PPO2 | |
| SEQ_ID_NO: 7 | DNA | Beta | Genomic DNA sequence of R126A mutant of | 37009 |
| | | vulgaris | Beta vulgaris PPO2 | |
| SEQ_ID_NO: 8 | CDNA | Beta | cDNA sequence of R126A mutant of Beta | 1575 |
| | | vulgaris | vulgaris PPO2 | |
| SEQ_ID_NO: 9 | protein | Beta | Protein sequence of R126A mutant of Beta | 524 |
| | | vulgaris | vulgaris PPO2 | |
| SEQ_ID_NO: 10 | DNA | Beta | Genomic DNA sequence of R126G mutant of | 37009 |
| | | vulgaris | Beta vulgaris PPO2 | |
| SEQ_ID_NO: 11 | CDNA | Beta | cDNA sequence of R126G mutant of Beta | 1575 |
| | | vulgaris | vulgaris PPO2 | |
| SEQ_ID_NO: 12 | protein | Beta | Protein sequence of R126G mutant of Beta | 524 |
| | | vulgaris | vulgaris PPO2 | |
| SEQ_ID_NO: 13 | DNA | Beta | Genomic DNA sequence of R126L mutant of | 37009 |
| | | vulgaris | Beta vulgaris PPO2 | |
| SEQ_ID_NO: 14 | CDNA | Beta | cDNA sequence of R126L mutant of Beta | 1575 |
| | | vulgaris | vulgaris PPO2 | |
| SEQ_ID_NO: 15 | protein | Beta | Protein sequence of R126L mutant of Beta | 524 |
| | | vulgaris | vulgaris PPO2 | |
| SEQ_ID_NO: 16 | DNA | Beta | Genomic DNA sequence of R126I mutant of | 37009 |
| | | vulgaris | Beta vulgaris PPO2 | |
| SEQ_ID_NO: 17 | CDNA | Beta | cDNA sequence of R126I mutant of Beta | 1575 |
| | | vulgaris | vulgaris PPO2 | |
| SEQ_ID_NO: 18 | protein | Beta | Protein sequence of R126I mutant of Beta | 524 |
| | | vulgaris | vulgaris PPO2 | |
| SEQ_ID_NO: 19 | DNA | Beta | Genomic DNA sequence of R126M mutant of | 37009 |
| | | vulgaris | Beta vulgaris PPO2 | |
| SEQ_ID_NO: 20 | CDNA | Beta | cDNA sequence of R126M mutant of Beta | 1575 |
| | | vulgaris | vulgaris PPO2 | |
| SEQ_ID_NO: 21 | protein | Beta | Protein sequence of R126M mutant of Beta | 524 |
| | | vulgaris | vulgaris PPO2 | |
| SEQ_ID_NO: 22 | DNA | Beta | Genomic DNA sequence of F420V mutant of | 37009 |
| | | vulgaris | Beta vulgaris PPO2 | |
| SEQ_ID_NO: 23 | CDNA | Beta | cDNA sequence of F420V mutant of Beta | 1575 |
| | | vulgaris | vulgaris PPO2 | |
| SEQ_ID_NO: 24 | protein | Beta | Protein sequence of F420V mutant of Beta | 524 |
| | | vulgaris | vulgaris PPO2 | |
| SEQ_ID_NO: 25 | DNA | Beta | Genomic DNA sequence of F420M mutant of | 37009 |
| | | vulgaris | Beta vulgaris PPO2 | |
| SEQ_ID_NO: 26 | cDNA | Beta | cDNA sequence of F420M mutant of Beta | 1575 |
| | | vulgaris | vulgaris PPO2 | |
| SEQ_ID_NO: 27 | protein | Beta | Protein sequence of F420M mutant of Beta | 524 |
| | | vulgaris | vulgaris PPO2 | |
| SEQ_ID_NO: 28 | DNA | Beta | Genomic DNA sequence of F420I mutant of | 37009 |
| | | vulgaris | Beta vulgaris PPO2 | |
| SEQ_ID_NO: 29 | CDNA | Beta | cDNA sequence of F420I mutant of Beta vulgaris | 1575 |
| | | vulgaris | PPO2 | |
| SEQ_ID_NO: 30 | protein | Beta | Protein sequence of F420I mutant of Beta | 524 |
| | | vulgaris | vulgaris PPO2 | |
| SEQ_ID_NO: 31 | DNA | Beta | Genomic DNA sequence of F420L mutant of | 37009 |
| | | vulgaris | Beta vulgaris PPO2 | |
| SEQ_ID_NO: 32 | cDNA | Beta | cDNA sequence of F420L mutant of Beta | 1575 |
| | | vulgaris | vulgaris PPO2 | |
| SEQ_ID_NO: 33 | protein | Beta | Protein sequence of F420L mutant of Beta | 524 |
| | | vulgaris | vulgaris PPO2 | |
| SEQ_ID_NO: 35 | DNA | Beta | Genomic DNA sequence of L397E mutant of | 37009 |
| | | vulgaris | Beta vulgaris PPO2 | |
| SEQ_ID_NO: 36 | CDNA | Beta | cDNA sequence of L397E mutant of Beta | 1575 |
| | | vulgaris | vulgaris PPO2 | |
| SEQ_ID_NO: 37 | protein | Beta | Protein sequence of L397E mutant of Beta | 524 |
| | | vulgaris | vulgaris PPO2 | |
| SEQ_ID_NO: 38 | DNA | Beta | Genomic DNA sequence of G398A mutant of | 37009 |
| | | vulgaris | Beta vulgaris PPO2 | |
| SEQ_ID_NO: 39 | cDNA | Beta | cDNA sequence of G398A mutant of Beta | 1575 |
| | | vulgaris | vulgaris PPO2 | |
| SEQ_ID_NO: 40 | protein | Beta | Protein sequence of G398A mutant of Beta | 524 |
| | | vulgaris | vulgaris PPO2 | |
| SEQ_ID_NO: 41 | DNA | Beta | Genomic DNA sequence of L397E + F420V | 37009 |
| | | vulgaris | double mutant of Beta vulgaris PPO2 | |
| SEQ_ID_NO: 42 | CDNA | Beta | cDNA sequence of L397E + F420V double | 1575 |
| | | vulgaris | mutant of Beta vulgaris PPO2 | |
| SEQ_ID_NO: 43 | protein | Beta | Protein sequence of L397E + F420V double | 524 |
| | | vulgaris | mutant of Beta vulgaris PPO2 | |
| SEQ_ID_NO: 44 | DNA | Beta | Genomic DNA sequence of R126L mutant of | 37009 |
| | | vulgaris | Beta vulgaris PPO2 | |
| SEQ_ID_NO: 45 | cDNA | Beta | cDNA sequence of R126L mutant of Beta | 1575 |
| | | vulgaris | vulgaris PPO2 | |
| SEQ_ID_NO: 46 | DNA | Beta | Genomic DNA sequence of F420L mutant of | 37009 |
| | | vulgaris | Beta vulgaris PPO2 | |
| SEQ_ID_NO: 47 | cDNA | Beta | cDNA sequence of F420L mutant of Beta | 1575 |
| | | vulgaris | vulgaris PPO2 | |
| SEQ_ID_NO: 97 | DNA | Beta | Genomic DNA sequence of G208/209 mutant of | 37006 |
| | | vulgaris | Beta vulgaris PPO2 with silent mutation | |
| SEQ_ID_NO: 98 | CDNA | Beta | cDNA sequence of G208/209 mutant of Beta | 1572 |
| | | vulgaris | vulgaris PPO2 | |
| SEQ_ID_NO: 99 | DNA | Beta | Genomic DNA sequence of G398A mutant of | 37009 |
| | | vulgaris | Beta vulgaris PPO2 with silent mutation | |
| SEQ_ID_NO: 100 | DNA | Beta | Genomic DNA (gDNA) sequence of L397E and | 37009 |
| | | vulgaris | F420V mutant of Beta vulgaris PPO2 with silent | |
| | | | mutation | |
| SEQ_ID_NO: 101 | cDNA | Beta | cDNA sequence of L397E and F420V mutant of | 1575 |
| | | vulgaris | Beta vulgaris PPO2 | |
| SEQ_ID_NO: 102 | DNA | Beta | Genomic DNA (gDNA) sequence of R126L | 37009 |
| | | vulgaris | mutant of Beta vulgaris PPO2 with silent | |
| | | | mutation | |
| SEQ_ID_NO: 103 | CDNA | Beta | cDNA sequence of R126L mutant of Beta | 1575 |
| | | vulgaris | vulgaris PPO2 | |
| SEQ_ID_NO: 104 | DNA | Beta | Genomic DNA (gDNA) sequence of G208/209 | 37006 |
| | | vulgaris | mutant of Beta vulgaris PPO2 with silent | |
| | | | mutation | |
| SEQ_ID_NO: 105 | cDNA | Beta | cDNA sequence of G208/209 mutant of Beta | 1572 |
| | | vulgaris | vulgaris PPO2 | |
| SEQ_ID_NO: 106 | DNA | Beta | Genomic DNA (gDNA) sequence of G398A | 37009 |
| | | vulgaris | mutant of Beta vulgaris PPO2 with silent | |
| | | | mutation | |
| SEQ_ID_NO: 107 | DNA | Beta | Genomic DNA (gDNA) sequence of L397E and | 37009 |
| | | vulgaris | F420V mutant of Beta vulgaris PPO2 with silent | |
| | | | mutation | |
| SEQ_ID_NO: 108 | cDNA | Beta | cDNA sequence of L397E and F420V mutant of | 1575 |
| | | vulgaris | Beta vulgaris PPO2 | |

### Example 12: Combining Different Mutations in Hybrids

To evaluate the resistance in lines carrying more than one PPO edit, plants produced by the methods above may be crossed to produce various cis (same allele) and trans (homologous allele) heterozygotes and homozygotes. As an example, flowering individuals of the male-fertile/female-fertile version of a line carrying the G209 or the G208 edit may be placed in an isolation chamber together with flowering individuals of the male-sterile/female-fertile version of a carrying the R126 edit. This is repeated using all genotypes and combination of edits. The seeds from the male-sterile/female-fertile plants were harvested as single hybrid seeds, and genetic edits can be confirmed using sequencing or marker analysis.

Additionally, as some edits may be produced on the same (cis) allele, a number of combinations are envisioned, including for example, wherein one edit may be in a homozygous state, and a second may be in a heterozygous state (i.e., a plant comprising a 397/420 double edit allele is crossed with a plant comprising a 420 edited allele to produce a plant homozygous for a substitution at 420 and heterozygous for a substitution at 397).

### Numbered Embodiments

1. A Beta vulgaris plant, or part thereof, comprising an engineered nucleic acid encoding a protoporphyrinogen oxidase 2 (PPO2) amino acid sequence, wherein said PPO2 amino acid sequence comprises an in-frame deletion of glycine at a position corresponding to 208 and/or 209 of SEQ ID NO: 3.

2. The Beta vulgaris plant or part thereof of embodiment 1, wherein the engineered nucleic acid encoding a protoporphyrinogen oxidase 2 (PPO2) amino acid sequence is obtained by targeted mutagenesis of the endogenous PPO2 gene.

3. The Beta vulgaris plant or part thereof of embodiment 1 or 2, wherein the deletion corresponds to position 208 of SEQ ID NO: 3.

4. The Beta vulgaris plant or part thereof of embodiment 1 or 2, wherein the deletion corresponds to position 209 of SEQ ID NO: 3.

5. The Beta vulgaris plant, or part thereof of any of embodiments 1-4, wherein the engineered PPO2 protein is at least 90% identical to SEQ ID NO: 3.

6. The Beta vulgaris plant, or part thereof of embodiment 3 or 4, wherein the engineered PPO2 protein is at least 98% identical to SEQ ID NO: 6.

7. The Beta vulgaris plant, or part thereof of any one of embodiments 1-5, wherein the engineered PPO2 protein is encoded by a nucleic acid having at least 90% identity to SEQ ID NO: 1.

8. The Beta vulgaris plant or part thereof of any one of embodiments 1-7, wherein the plant, or part thereof further comprises a nucleic acid encoding a protoporphyrinogen oxidase 2 (PPO2) amino acid sequence having at least one mutation selected from:
a) a substitution of arginine at a position corresponding to 126 of SEQ ID NO: 3;
b) a substitution of leucine at a position corresponding to 397 of SEQ ID NO: 3;
c) a substitution of glycine at a position corresponding to 398 of SEQ ID NO: 3; and
d) a substitution of phenylalanine at a position corresponding to 420 of SEQ ID NO: 3.

9. The Beta vulgaris plant or part thereof of embodiment 8, wherein the arginine at position 126 is replaced with alanine, glycine, leucine, isoleucine, or methionine.

10. The Beta vulgaris plant or part thereof of embodiment 8, wherein leucine at position 397 is replaced with glutamic acid.

11. The Beta vulgaris plant or part thereof of embodiment 8, wherein glycine at position 398 is replaced with alanine.

12. The Beta vulgaris plant or part thereof of embodiment 8, wherein phenylalanine at position 420 is replaced with valine, methionine, isoleucine, or leucine.

13. The Beta vulgaris plant or part thereof of embodiment 8, wherein leucine at position 397 is replaced with glutamic acid and the phenylalanine at position 420 is replaced with valine.

14. The Beta vulgaris plant or part thereof of any one of embodiments 8-13, wherein the nucleic acid encoding a protoporphyrinogen oxidase 2 (PPO2) amino acid sequence having at least one of a), b), c), and d) is on the same allele as the deletion of glycine at a position corresponding to 208 and/or 209 of SEQ ID NO: 3.

15. The Beta vulgaris plant or part thereof of any one of embodiments 8-13, wherein the nucleic acid encoding a protoporphyrinogen oxidase 2 (PPO2) amino acid sequence having at least one of a), b), c), and d) is on a different allele as the deletion of glycine at a position corresponding to 208 and/or 209 of SEQ ID NO: 3.

16. The Beta vulgaris plant, or part thereof, of any one of embodiments 1-15, wherein the plant or plant part is homozygous for at least one of:
a) a substitution of arginine at a position corresponding to 126 of SEQ ID NO: 3;
b) an in-frame deletion of glycine at a position corresponding to 208 and/or 209 of SEQ ID NO: 3;
c) a substitution of leucine at a position corresponding to 397 of SEQ ID NO: 3;
d) a substitution of glycine at a position corresponding to 398 of SEQ ID NO: 3; and
e) a substitution of phenylalanine at a position corresponding to 420 of SEQ ID NO: 3.

17. The Beta vulgaris plant, or part thereof, of any one of embodiments 1-16, wherein the plant or part thereof comprises:
a) one allele having the deletion of any one of embodiments 1-7; and
b) a second allele having the mutation of any one of embodiments 8-13.

18. The Beta vulgaris plant, or part thereof, of embodiment 17, wherein the plant or part thereof is produced by crossing a first Beta vulgaris plant which is homozygous for the deletion of any one of embodiments 1-7 with a second Beta vulgaris plant which is homozygous for the mutation of any one of embodiments 8-13.

19. The Beta vulgaris plant, or part thereof of any one of embodiments 1-18, wherein the plant or part thereof is resistant to an herbicide.

20. The Beta vulgaris plant, or part thereof of embodiment 19, wherein the plant or part thereof has increased tolerance to an herbicide that inhibits protoporphyrinogen oxidase when compared to a plant not having the in-frame amino acid deletion at a position corresponding to 208 and/or 209 of SEQ ID NO: 3.

21. The Beta vulgaris plant, or part thereof of embodiment 20, wherein the herbicide is selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

22. The Beta vulgaris plant, or part thereof of any one of embodiments 1-21, wherein the plant is a sugar beet or a fodder beet.

23. The Beta vulgaris plant, or part thereof of any one of embodiments 1-22, further comprising an additional desired trait.

24. The Beta vulgaris plant, or part thereof of embodiment 23, wherein the desired trait confers resistance to a non-PPO herbicide.

25. The Beta vulgaris plant, or part thereof of embodiment 24, wherein the non-PPO herbicide is a glyphosate.

26. The Beta vulgaris plant, or part thereof, of embodiment 25, wherein the glyphosate resistance is conferred by the H7-1 event.

27. An isolated polynucleotide comprising an engineered nucleic acid sequence encoding a protein comprising an in-frame amino acid deletion at a position corresponding to 208 and/or 209 of SEQ ID NO: 3.

28. The polynucleotide of embodiment 27, wherein the engineered nucleic acid sequence is at least 90% identical to SEQ ID NO: 1.

29. The polynucleotide of embodiment 27, wherein the engineered nucleic acid sequence further comprises one or more additional mutations encoding amino acid substitutions at positions corresponding to 126, 397, 398, and 420 of SEQ ID NO: 3.

30. A DNA construct comprising the polynucleotide of any one of embodiments 27-29.

31. A method for producing a Beta vulgaris plant or plant cell having an engineered PPO2 protein comprising: introducing a nucleic acid mutation by targeted genome editing that results in an in-frame amino acid deletion corresponding to position 208 and/or 209 of SEQ ID NO: 3.

32. The method of embodiment 31, wherein the targeted genome editing is a CRISPR/Cas system.

33. The method of embodiment 32, wherein the CRISPR/Cas system comprises:
a) a guide RNA sequence comprising SEQ ID NO: 79, or an expression construct encoding said guide RNA;
b) a donor template sequence selected from SEQ ID NO: 60 and 90; and
c) a Cas9 DNA nuclease, or an expression construct encoding said nuclease.

34. The method of embodiment 32, wherein the CRISPR/Cas system comprises:
a) a guide RNA sequence selected from SEQ ID NOs: 75-76, or an expression construct encoding said guide RNA;
b) a donor template sequence selected from SEQ ID NOs: 51, 63 and 94; and
c) a Cpf1 DNA nuclease, or an expression construct encoding said nuclease.

35. The method of any one of embodiments 31-34, wherein the Beta vulgaris plant or plant cell further comprises a nucleic acid encoding a protoporphyrinogen oxidase 2 (PPO2) amino acid sequence having at least one of:
a) a substitution of arginine at a position corresponding to 126 of SEQ ID NO: 3;
b) a substitution of leucine at a position corresponding to 397 of SEQ ID NO: 3;
c) a substitution of glycine at a position corresponding to 398 of SEQ ID NO: 3; and
d) a substitution of phenylalanine at a position corresponding to 420 of SEQ ID NO: 3.

36. The method of embodiment 35, wherein the nucleic acid encoding a protoporphyrinogen oxidase 2 (PPO2) amino acid sequence having at least one of a), b), c), and d) is on the same allele as the deletion corresponding to position number 208 and/or 209 of SEQ ID NO: 3.

37. The method of embodiment 35, wherein the nucleic acid encoding a protoporphyrinogen oxidase 2 (PPO2) amino acid sequence having at least one of a), b), c), and d) is on a different allele as the deletion corresponding to position number 208 and/or 209 of SEQ ID NO: 3.

38. The method of embodiment 37, wherein the nucleic acid encoding a protoporphyrinogen oxidase 2 (PPO2) amino acid sequence having at least one of a), b), c), and d) on a different allele is introduced by a breeding technique.

39. A Beta vulgaris plant produced by the method of any one of embodiments 31-38, wherein said plant is resistant or tolerant to one or more herbicides.

40. The Beta vulgaris plant of embodiment 39, wherein the plant is resistant to a PPO herbicide selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

41. A method for controlling undesired vegetation at a Beta vulgaris cultivation site, the method comprising:
a) growing the Beta vulgaris plant of any one of embodiments 19, 20, 21, 39, or 40 at a cultivation site; and
b) applying to the cultivation site an effective amount of a PPO herbicide.

42. The method of embodiment 41, wherein the PPO herbicide selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

43. A guide RNA suitable for use in a CRISPR based genome editing system, wherein said guide RNA is selected from SEQ ID NOs: 67-80.

44. A donor template sequence suitable for use in a CRISPR based genome editing system, wherein said donor template sequence is selected from SEQ ID NOs: 48-66 and 90-96.

45. A DNA construct comprising the guide RNA of embodiment 43 and the donor template sequence of embodiment 44.

46. An engineered PPO2 protein comprising an in-frame amino acid deletion corresponding to position number 208 and/or 209 in SEQ ID NO: 3.

47. The engineered protein of embodiment 46, wherein said protein is at least 90% identical to SEQ ID NO: 3.

48. The engineered protein of embodiment 46, wherein said protein is at least 98% identical to SEQ ID NO: 6.

49. The engineered protein of any one of embodiments 46-48, wherein said protein further comprises at least one of:
a) a substitution of arginine at a position corresponding to 126 of SEQ ID NO: 3;
b) a substitution of leucine at a position corresponding to 397 of SEQ ID NO: 3;
c) a substitution of glycine at a position corresponding to 398 of SEQ ID NO: 3; and
d) a substitution of phenylalanine at a position corresponding to 420 of SEQ ID NO: 3.

50. A method of producing a Beta vulgaris plant with increased tolerance to an herbicide that inhibits protoporphyrinogen oxidase comprising the steps of:
a) transfecting a protoplast obtained from Beta vulgaris cells with a genome editing system to generate a transfected protoplast, wherein the genome editing system comprises: i) a Cas enzyme;
   ii) at least one guide RNA (gRNA), wherein the at least one gRNA targets a genomic region corresponding to between position 5457 and 5502 of SEQ ID NO: 1; and
   iii) at least one single-stranded donor DNA repair template designed to introduce a deletion of glycine at a position corresponding to 208 and/or 209 of SEQ ID NO: 3;
b) exposing the transfected protoplast to a selective pressure of at least one herbicide that inhibits protoporphyrinogen oxidase;
c) selecting a protoplast comprising a deletion of glycine at a position corresponding to 208 and/or 209 of SEQ ID NO: 3; and
d) regenerating a plant from said selected protoplast to produce a Beta vulgaris plant with increased tolerance to an herbicide that inhibits protoporphyrinogen oxidase.

51. A method of detecting an in-frame deletion of glycine at a position corresponding to 208 and/or 209 of SEQ ID NO: 3 in a Beta vulgaris plant or part thereof, comprising:
obtaining a Beta vulgaris plant or part thereof, and
analyzing the Beta vulgaris plant or part thereof using at least one of SEQ ID NOs: 112-122 to detect an in-frame deletion of glycine at a position corresponding to 208 and/or 209 of SEQ ID NO: 3.

52. The method of embodiment 51, wherein the analyzing comprises using at least one of SEQ ID NOs: 112-120 in a Kompetitive allele specific PCR assay.

53. The method of embodiment 51 or 52, wherein the analyzing comprises using at least one of SEQ ID NO: 113, SEQ ID NO: 115, SEQ ID NO: 117, SEQ ID NO: 118, SEQ ID NO: 119, and SEQ ID NO: 120.

54. The method of embodiment 51, wherein the analyzing comprises using at least one of SEQ ID NO: 121 and SEQ ID NO: 122 in a chain termination sequencing method.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

What is claimed is:

1. A Beta vulgaris plant, or part thereof, comprising an engineered nucleic acid encoding a protoporphyrinogen oxidase 2 (PPO2) amino acid sequence, wherein said PPO2 amino acid sequence comprises an in-frame deletion of glycine at a position corresponding to 208 and/or 209 of SEQ ID NO: 3.

2. The Beta vulgaris plant or part thereof of claim 1, wherein the engineered nucleic acid encoding a protoporphyrinogen oxidase 2 (PPO2) amino acid sequence is obtained by targeted mutagenesis of the endogenous PPO2 gene.

3. The Beta vulgaris plant or part thereof of claim 1, wherein the deletion corresponds to position 208 of SEQ ID NO: 3.

4. The Beta vulgaris plant or part thereof of claim 1, wherein the deletion corresponds to position 209 of SEQ ID NO: 3.

5. The Beta vulgaris plant, or part thereof of claim 1, wherein the engineered PPO2 protein is at least 90% identical to SEQ ID NO: 3.

6. The Beta vulgaris plant, or part thereof of claim 1, wherein the engineered PPO2 protein is at least 98% identical to SEQ ID NO: 6.

7. The Beta vulgaris plant, or part thereof of claim 1, wherein the engineered PPO2 protein is encoded by a nucleic acid having at least 90% identity to SEQ ID NO: 1.

8. The Beta vulgaris plant or part thereof of claim 1, wherein the plant, or part thereof further comprises a nucleic acid encoding a protoporphyrinogen oxidase 2 (PPO2) amino acid sequence having at least one mutation selected from:
a) a substitution of arginine at a position corresponding to 126 of SEQ ID NO: 3;
b) a substitution of leucine at a position corresponding to 397 of SEQ ID NO: 3;
c) a substitution of glycine at a position corresponding to 398 of SEQ ID NO: 3; and
d) a substitution of phenylalanine at a position corresponding to 420 of SEQ ID NO: 3.

9. The Beta vulgaris plant or part thereof of claim 8, wherein the arginine at position 126 is replaced with alanine, glycine, leucine, isoleucine, or methionine.

10. The Beta vulgaris plant or part thereof of claim 8, wherein leucine at position 397 is replaced with glutamic acid.

11. The Beta vulgaris plant or part thereof of claim 8, wherein glycine at position 398 is replaced with alanine.

12. The Beta vulgaris plant or part thereof of claim 8, wherein phenylalanine at position 420 is replaced with valine, methionine, isoleucine, or leucine.

13. The Beta vulgaris plant or part thereof of claim 8, wherein leucine at position 397 is replaced with glutamic acid and the phenylalanine at position 420 is replaced with valine.

14. The Beta vulgaris plant or part thereof of claim 8, wherein the nucleic acid encoding a protoporphyrinogen oxidase 2 (PPO2) amino acid sequence having at least one of a), b), c), and d) is on the same allele as the deletion of glycine at a position corresponding to 208 and/or 209 of SEQ ID NO: 3.

15. The Beta vulgaris plant or part thereof of claim 8, wherein the nucleic acid encoding a protoporphyrinogen oxidase 2 (PPO2) amino acid sequence having at least one of a), b), c), and d) is on a different allele as the deletion of glycine at a position corresponding to 208 and/or 209 of SEQ ID NO: 3.

16. The Beta vulgaris plant, or part thereof, of claim 1, wherein the plant or plant part is homozygous for at least one of:
a) a substitution of arginine at a position corresponding to 126 of SEQ ID NO: 3;
b) an in-frame deletion of glycine at a position corresponding to 208 and/or 209 of SEQ ID NO: 3;
c) a substitution of leucine at a position corresponding to 397 of SEQ ID NO: 3;
d) a substitution of glycine at a position corresponding to 398 of SEQ ID NO: 3; and
e) a substitution of phenylalanine at a position corresponding to 420 of SEQ ID NO: 3.

17. The Beta vulgaris plant, or part thereof, of claim 1, wherein the plant or part thereof comprises:
a) one allele having the deletion of claim 1; and
b) a second allele having the mutation of claim 8.

18. The Beta vulgaris plant, or part thereof, of claim 17, wherein the plant or part thereof is produced by crossing a first Beta vulgaris plant which is homozygous for the deletion of claim 1 with a second Beta vulgaris plant which is homozygous for the mutation of claim 8.

19. The Beta vulgaris plant, or part thereof of claim 1, wherein the plant or part thereof is resistant to an herbicide.

20. The Beta vulgaris plant, or part thereof of claim 19, wherein the plant or part thereof has increased tolerance to an herbicide that inhibits protoporphyrinogen oxidase when compared to a plant not having the in-frame amino acid deletion at a position corresponding to 208 and/or 209 of SEQ ID NO: 3.

21. The Beta vulgaris plant, or part thereof of claim 20, wherein the herbicide is selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

22. The Beta vulgaris plant, or part thereof of claim 1, wherein the plant is a sugar beet or a fodder beet.

23. The Beta vulgaris plant, or part thereof of claim 1, further comprising an additional desired trait.

24. The Beta vulgaris plant, or part thereof of claim 23, wherein the desired trait confers resistance to a non-PPO herbicide.

25. The Beta vulgaris plant, or part thereof of claim 24, wherein the non-PPO herbicide is a glyphosate.

26. The Beta vulgaris plant, or part thereof, of claim 25, wherein the glyphosate resistance is conferred by the H7-1 event.

27. An isolated polynucleotide comprising an engineered nucleic acid sequence encoding a protein comprising an in-frame amino acid deletion at a position corresponding to 208 and/or 209 of SEQ ID NO: 3.

28. The polynucleotide of claim 27, wherein the engineered nucleic acid sequence is at least 90% identical to SEQ ID NO: 1.

29. The polynucleotide of claim 27, wherein the engineered nucleic acid sequence further comprises one or more additional mutations encoding amino acid substitutions at positions corresponding to 126, 397, 398, and 420 of SEQ ID NO: 3.

30. A DNA construct comprising the polynucleotide of claim 27.

31. A method for producing a Beta vulgaris plant or plant cell having an engineered PPO2 protein comprising: introducing a nucleic acid mutation by targeted genome editing that results in an in-frame amino acid deletion corresponding to position 208 and/or 209 of SEQ ID NO: 3.

32. The method of claim 31, wherein the targeted genome editing is a CRISPR/Cas system.

33. The method of claim 32, wherein the CRISPR/Cas system comprises:
a) a guide RNA sequence comprising SEQ ID NO: 79, or an expression construct encoding said guide RNA;
d) a donor template sequence selected from SEQ ID NO: 60 and 90; and
e) a Cas9 DNA nuclease, or an expression construct encoding said nuclease.

34. The method of claim 32, wherein the CRISPR/Cas system comprises:
a) a guide RNA sequence selected from SEQ ID NOs: 75-76, or an expression construct encoding said guide RNA;
d) a donor template sequence selected from SEQ ID NOs: 51, 63 and 94; and
e) a Cpf1 DNA nuclease, or an expression construct encoding said nuclease.

35. The method of claim 31, wherein the Beta vulgaris plant or plant cell further comprises a nucleic acid encoding a protoporphyrinogen oxidase 2 (PPO2) amino acid sequence having at least one of:
a) a substitution of arginine at a position corresponding to 126 of SEQ ID NO: 3;
b) a substitution of leucine at a position corresponding to 397 of SEQ ID NO: 3;
c) a substitution of glycine at a position corresponding to 398 of SEQ ID NO: 3; and
d) a substitution of phenylalanine at a position corresponding to 420 of SEQ ID NO: 3.

36. The method of claim 35, wherein the nucleic acid encoding a protoporphyrinogen oxidase 2 (PPO2) amino acid sequence having at least one of a), b), c), and d) is on the same allele as the deletion corresponding to position number 208 and/or 209 of SEQ ID NO: 3.

37. The method of claim 35, wherein the nucleic acid encoding a protoporphyrinogen oxidase 2 (PPO2) amino acid sequence having at least one of a), b), c), and d) is on a different allele as the deletion corresponding to position number 208 and/or 209 of SEQ ID NO: 3.

38. The method of claim 37, wherein the nucleic acid encoding a protoporphyrinogen oxidase 2 (PPO2) amino acid sequence having at least one of a), b), c), and d) on a different allele is introduced by a breeding technique.

39. A Beta vulgaris plant produced by the method of claim 31, wherein said plant is resistant or tolerant to one or more herbicides.

40. The Beta vulgaris plant of claim 39, wherein the plant is resistant to a PPO herbicide selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

41. A method for controlling undesired vegetation at a Beta vulgaris cultivation site, the method comprising:
a) growing the Beta vulgaris plant of any one of claims 19, 20, 21, 39, or 40 at a cultivation site; and
b) applying to the cultivation site an effective amount of a PPO herbicide.

42. The method of claim 41, wherein the PPO herbicide selected from the group consisting of: acifluorfen, fomesafen, lactofen, fluoroglycofen-ethyl, oxyfluorfen, flumioxazin, azafenidin, carfentrazone-ethyl, sulfentrazone, fluthiacet-methyl, oxadiargyl, oxadiazon, pyraflufen-ethyl, saflufenacil, trifludimoxazin, and S-3100.

43. A guide RNA suitable for use in a CRISPR based genome editing system, wherein said guide RNA is selected from SEQ ID NOs: 67-80.

44. A donor template sequence suitable for use in a CRISPR based genome editing system, wherein said donor template sequence is selected from SEQ ID NOs: 48-66 and 90-96.

45. A DNA construct comprising the guide RNA of claim 43 and the donor template sequence of claim 44.

46. An engineered PPO2 protein comprising an in-frame amino acid deletion corresponding to position number 208 and/or 209 in SEQ ID NO: 3.

47. The engineered protein of claim 46, wherein said protein is at least 90% identical to SEQ ID NO: 3.

48. The engineered protein of claim 46, wherein said protein is at least 98% identical to SEQ ID NO: 6.

49. The engineered protein of claim 46, wherein said protein further comprises at least one of:
a) a substitution of arginine at a position corresponding to 126 of SEQ ID NO: 3;
b) a substitution of leucine at a position corresponding to 397 of SEQ ID NO: 3;
c) a substitution of glycine at a position corresponding to 398 of SEQ ID NO: 3; and
d) a substitution of phenylalanine at a position corresponding to 420 of SEQ ID NO: 3.

50. A method of producing a Beta vulgaris plant with increased tolerance to an herbicide that inhibits protoporphyrinogen oxidase comprising the steps of:
a) transfecting a protoplast obtained from Beta vulgaris cells with a genome editing system to generate a transfected protoplast, wherein the genome editing system comprises: i) a Cas enzyme;
ii) at least one guide RNA (gRNA), wherein the at least one gRNA targets a genomic region corresponding to between position 5457 and 5502 of SEQ ID NO: 1; and
iii) at least one single-stranded donor DNA repair template designed to introduce a deletion of glycine at a position corresponding to 208 and/or 209 of SEQ ID NO: 3;
b) exposing the transfected protoplast to a selective pressure of at least one herbicide that inhibits protoporphyrinogen oxidase;
c) selecting a protoplast comprising a deletion of glycine at a position corresponding to 208 and/or 209 of SEQ ID NO: 3; and
d) regenerating a plant from said selected protoplast to produce a Beta vulgaris plant with increased tolerance to an herbicide that inhibits protoporphyrinogen oxidase.

51. A method of detecting an in-frame deletion of glycine at a position corresponding to 208 and/or 209 of SEQ ID NO: 3 in a Beta vulgaris plant or part thereof, comprising:
obtaining a Beta vulgaris plant or part thereof, and
analyzing the Beta vulgaris plant or part thereof using at least one of SEQ ID NOs: 112-122 to detect an in-frame deletion of glycine at a position corresponding to 208 and/or 209 of SEQ ID NO: 3.

52. The method of claim 51, wherein the analyzing comprises using at least one of SEQ ID NOs: 112-120 in a Kompetitive allele specific PCR assay.

53. The method of claim 51 or 52, wherein the analyzing comprises using at least one of SEQ ID NO: 113, SEQ ID NO: 115, SEQ ID NO: 117, SEQ ID NO: 118, SEQ ID NO: 119, and SEQ ID NO: 120.

54. The method of claim 51, wherein the analyzing comprises using at least one of SEQ ID NO: 121 and SEQ ID NO: 122 in a chain termination sequencing method.
